(19) United States
(12) Patent Application Publication
(10) Pub. No.: US 2025/0025094 A1
(43) Pub. Date: Jan. 23, 2025

(54) INSTRUMENT AND MODEL FOR ROTATIONAL TRAUMATIC BRAIN INJURY

(71) Applicants: Arizona Board of Regents on Behalf of the University of Arizona, Tucson, AZ (US); The UAB Research Foundation, Birmingham, AL (US)

(72) Inventors: James Bibb, Phoenix, AZ (US); Alan Umfress, New York, NY (US)

(21) Appl. No.: 18/780,047

(22) Filed: Jul. 22, 2024

Related U.S. Application Data
(60) Provisional application No. 63/514,910, filed on Jul. 21, 2023.

(51) Int. Cl. A61B 5/00; A01K 1/03

(52) U.S. Cl. CPC A61B 5/4064; A01K 1/031; A61B 5/6803; A61B 2503/40

(57) ABSTRACT

A model and testing device for creating repetitive, rotational head trauma in rodents is disclosed herein. The testing device induces angular rotational acceleration and deceleration forces that result in injury or insult to the integrity of the intracranial tissues of the brain for the test animal and can be used to characterize, test the effects of, and/or investigate a therapeutic agent for rotational traumatic brain injury. In some embodiments, the testing device contains an animal restraint that is configured to secure the animal therein, while allowing the animal's head to rotate about the coronal plane during and following impact. The testing device can include an animal restraint, a restraint mount, configured to receive the animal restraint, a helmet assembly attached to the subject restraint mount, such that the helmet assembly is in pivotal rotation about a central axis of rotation, a pendulum arm, and an impact fixture.

### CROSS REFERENCE TO RELATED APPLICATION

This application claims benefit of and priority to U.S. Provisional Application No. 63/514,910, filed Jul. 21, 2023, which is incorporated by reference herein in its entirety.

### FIELD OF THE INVENTION

The disclosed invention is generally in the field of testing devices and models, particularly in the area of traumatic brain injury.

### BACKGROUND OF THE INVENTION

Millions of traumatic brain injuries (TBIs) occur annually. TBIs commonly result from falls, traffic accidents, and sports-related injuries, all of which involve rotational acceleration/deceleration of the brain. During these injuries, the brain endures a multitude of primary insults including compression of brain tissue, damaged vasculature, and diffuse axonal injury. All of these deleterious effects can contribute to secondary brain ischemia, cellular death, and neuroinflammation that progress for weeks, months, and lifetime after injury.

While the linear effects of head trauma have been extensively modeled, less is known about how rotational injuries mediate neuronal damage following injury.

Rotational TBI remains the most clinically relevant form of brain trauma [62]. However, understanding of these injuries has been limited by the lack of rotational acceleration-induced injury models in rodents that accurately recapitulate patient phenotypes.

Specifically, most preclinical rotational head trauma has been limited to large animal models due the conserved gyrencephalic brain structures lacking in rodent models [63]. While these large animal models of brain injury offer some physiologically relevant advantages, they remain costly and underpowered for genetic manipulations and preclinical screening of therapeutics [64]. The fundamental challenge with rodent models of rTBI has been to scale rotational forces to small brain structures.

Helmet telemetry data in humans have determined a typical sports-related head trauma induces rotational accelerations between 1-3 Krad/s² [30, 65]. Scaling to the 2 gram rat brain, these equate to rotational forces greater than 350 Krad/s² [31, 66].

Previously, rodent models including the Medical College of Wisconsin (MCW) and Closed-Head Impact Model of Engineered Rotational Acceleration (CHIMERA) have provided insights into modeling these injuries in small mammals. These models have demonstrated histopathological and behavioral abnormalities following injury and show differential outcomes in these injury models in comparison to blast models of TBI [67-69].

However, these models have remained limited largely to initial characterizations and, in some instances, lack equivalent scalar angular forces.

Therefore, there is a need for improved small animal models for rotational traumatic brain injury. There also remains a need for improved devices for use in modeling rotational traumatic brain injury in small animals and for testing possible interventions and treatments, which facilitates translation to humans.

It is an object of the invention to provide an improved small animal model for use in modeling rotational traumatic brain injury.

It is a further object of the invention to provide an improved testing device for use in modeling and/or testing rotational traumatic brain injury in mammals.

It is a further object of the invention to provide an improved testing device for use in testing the effects of rotational traumatic brain injury in mammals.

It is a further object of the invention to provide an improved testing device for use in testing potential interventions and/or treatments of rotational traumatic brain injury in mammals.

### SUMMARY OF THE INVENTION

A model and testing device for creating repetitive, rotational head trauma in rodents is disclosed herein. The testing device induces angular rotational acceleration and deceleration forces that result in injury or insult to the integrity of the intracranial tissues of the brain for the test animal. The deleterious effects of this include shearing and tearing of axonal tracks, compression of brain tissue comprised of various cell types and critical substructures including synapses, dendrites, axons, white matter, grey matter, and neurovasculature. These injuries may lead to sequela including neuronal injury and death, neuroinflammation, compromise in neurovasculature, disruption of the blood brain barrier, and chronic conditions such as loss of cognitive function, impaired mood, light sensitivity, headaches, balance impairment, edema, and neurodengenerative disorders including tauopathies and chronic traumatic encephalopathy.

In use, the model demonstrates acute and prolonged pathological, behavioral, and electrophysiological effects of rotational TBI (rTBI). The model and testing device can be used to understand the mechanisms of rTBI and how they may be effectively treated.

The testing device described herein contains an animal restraint that is configured to secure the animal therein, while allowing the animal's head to rotate about the coronal plane during and following impact. The testing device can include an animal restraint, a restraint mount, configured to receive the animal restraint, a helmet assembly attached to the subject restraint mount, such that the helmet assembly is in pivotal rotation about a central axis of rotation, a pendulum arm, and an impact fixture.

The testing device and method can be used to characterize, test the effects of, and/or investigate a therapeutic agent for rotational traumatic brain injury.

Using the testing device described herein, aberrant Cyclin-dependent kinase 5 (Cdk5) activity was determined to be a principal mediator of rTBI. As demonstrated in the examples, the device described herein was also used with Cdk5-enriched phosphoproteomics to uncover potential downstream mediators of rTBI and show pharmacological inhibition of Cdk5 reduces the cognitive and pathological consequences of injury.

BRIEF DESCRIPTIONS OF THE DRAWINGS

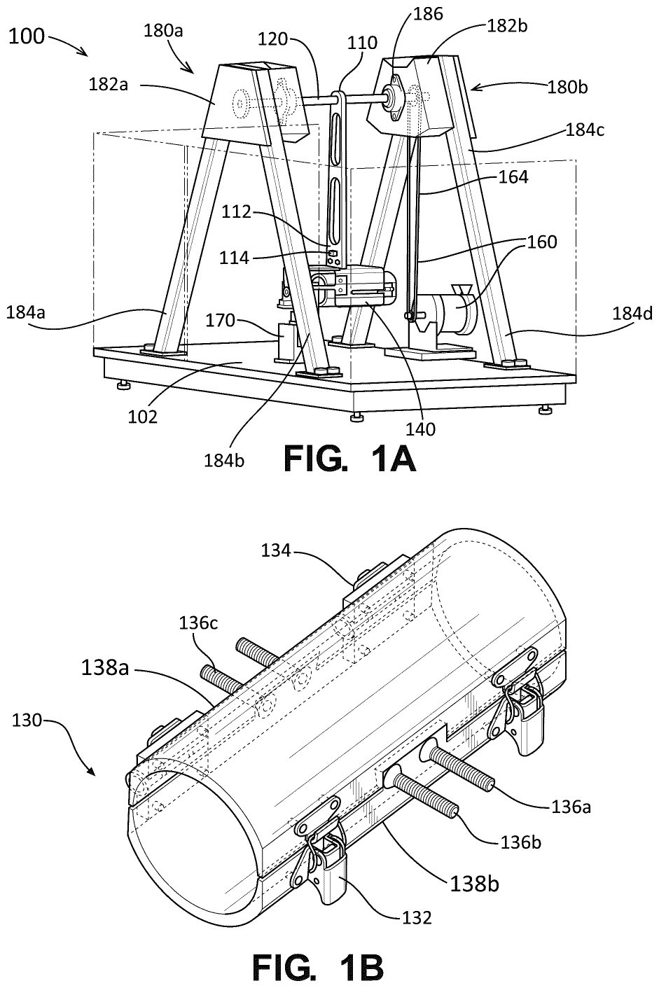

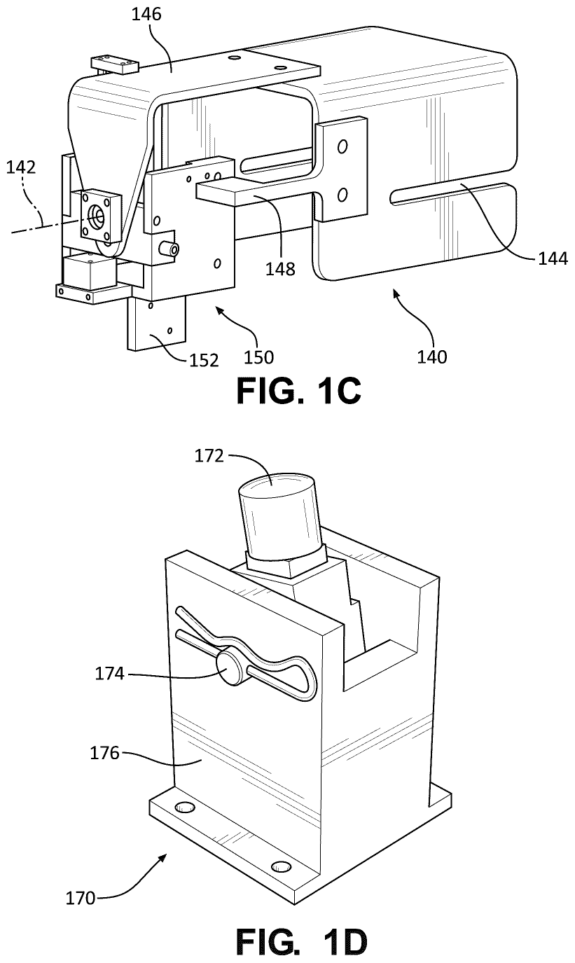

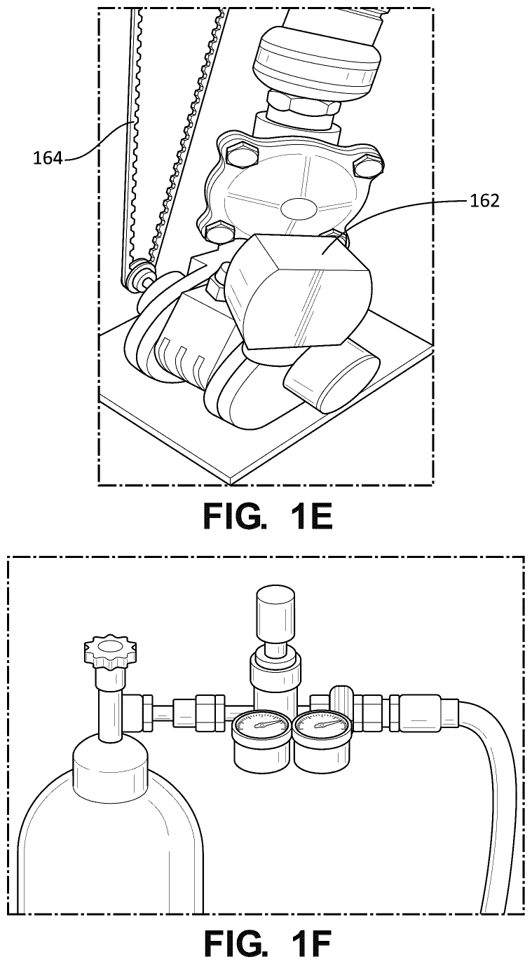

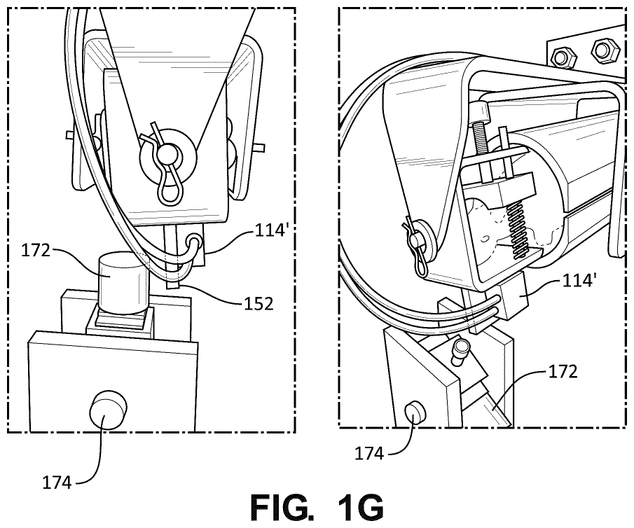

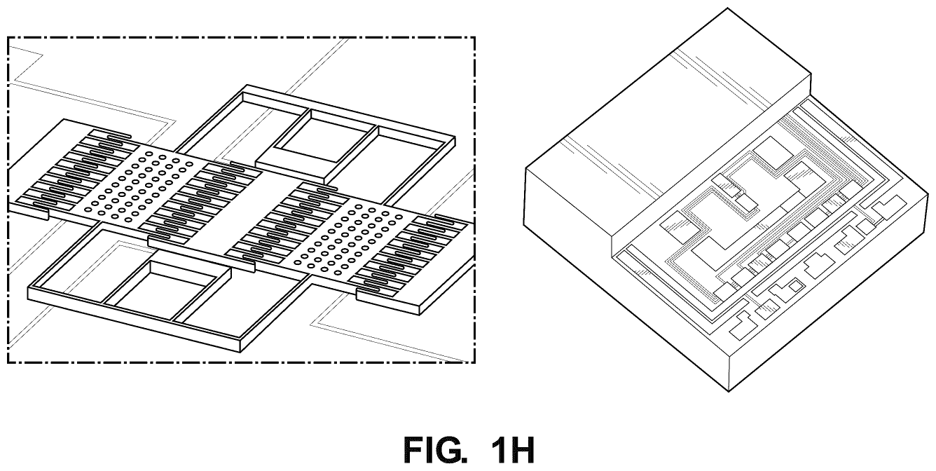

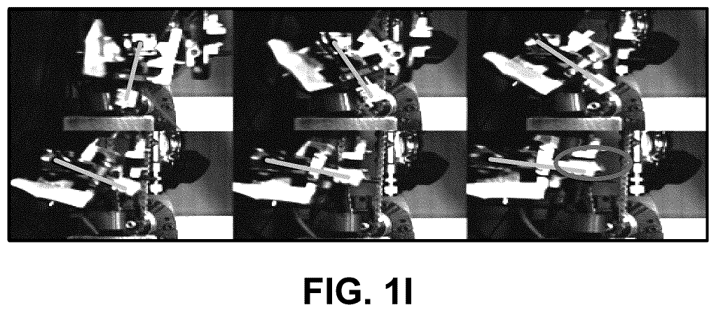

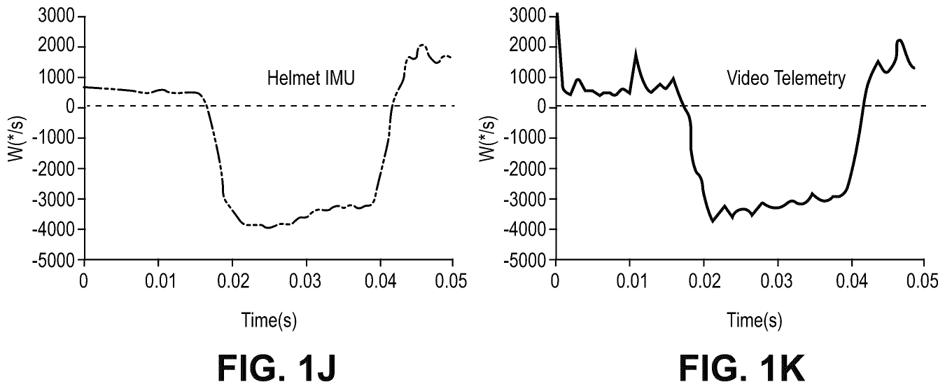

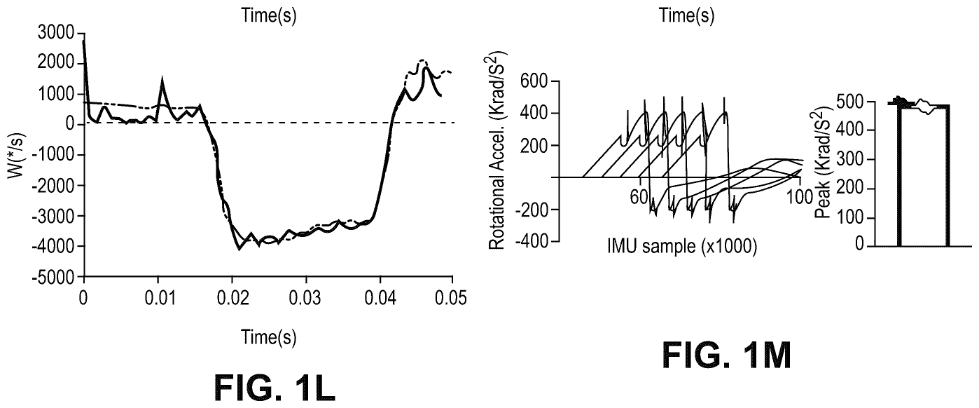

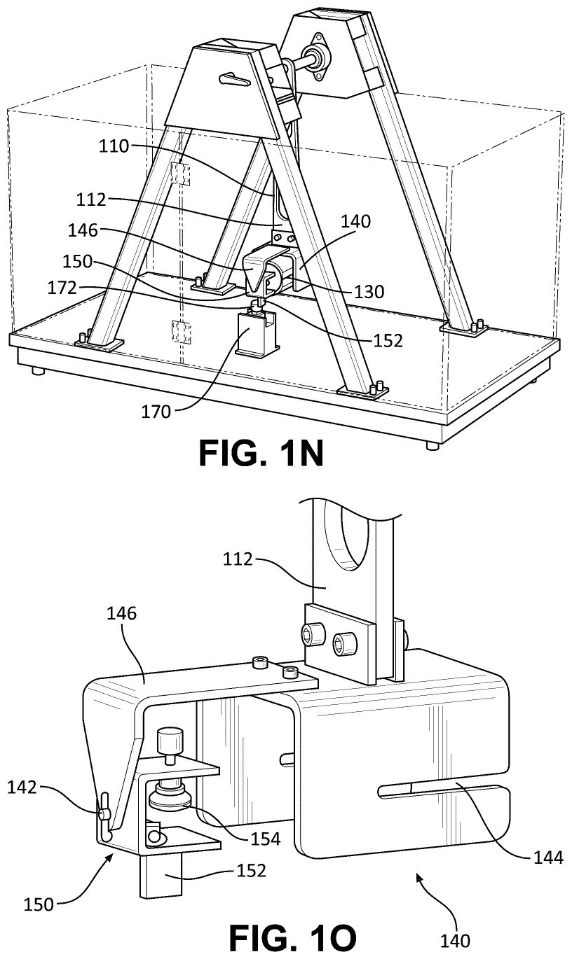

FIG. 1A is a schematic showing an exemplary head injury testing device for use with rodents.

FIG. 1B shows an exemplary animal restraint for use in the head injury testing device depicted in FIG. 1A.

FIG. 1C shows an exemplary restraint mount with a helmet system attached thereto at a helmet rotation joint, which allows the helmet system is able to rotate when impacted by a force. Typically, the restraint mount is fixedly attached to the end of the pendulum of the testing device depicted in FIG. 1A, and allows for the insertion of the animal restraint depicted in FIG. 1B and the removal of the animal restraint therefrom.

FIG. 1D shows an exemplary impact fixture, configured to impact the ventral strike plate of the helmet system to which a rodent's head is attached with the impact bumper when the testing device is in use.

FIG. 1E is a depiction of an exemplary pneumatic-chain drive system. FIG. 1F is a depiction of an exemplary pneumatic pressure system.

FIG. 1G (left) is an image of the strike plate just prior to impact with the impact bumper, which is located in a first position to facilitate impact. FIG. 1G (right) is a side view of helmet assembly and restraint with artificial rat inserted therein, one or more sensors, such as a helmet IMU is attached to the strike plate.

FIG. 1H is a depiction of a Model 633 6DOF inertia measurement unit, showing its oscillating variable capacitance gyro sensor combs (right) and a half cover removed view of IMU showing acceleration sensor, mass, supports, and circuitry (left).

FIG. 11 depicts an example of temporally sequential high-speed camera recording frames used to derive acceleration rates.

FIG. 1J is an exemplary recording from IMU, depicted graphically as angular speed W (°/s) versus time (seconds).

FIG. 1K is an exemplary graph based on video telemetry data, which was used to calculate angular speed W (°/s) versus time (seconds).

FIG. 1L is a graph providing an overlay of helmet IMU and video telemetry angular speeds (°/s) as a function of time (seconds).

FIG. 1M is a graph of Labview-derived rotational accelerations (Krad/s²) of 5 trial runs (left). Peak rotational accelerations for each run with standard error are also shown (right).

FIG. 1N is a schematic showing another exemplary head injury testing device for use with rodents without a pneumatic chain drive system.

FIG. 1O shows another exemplary restraint mount with a helmet system attached thereto via a helmet rotation joint.

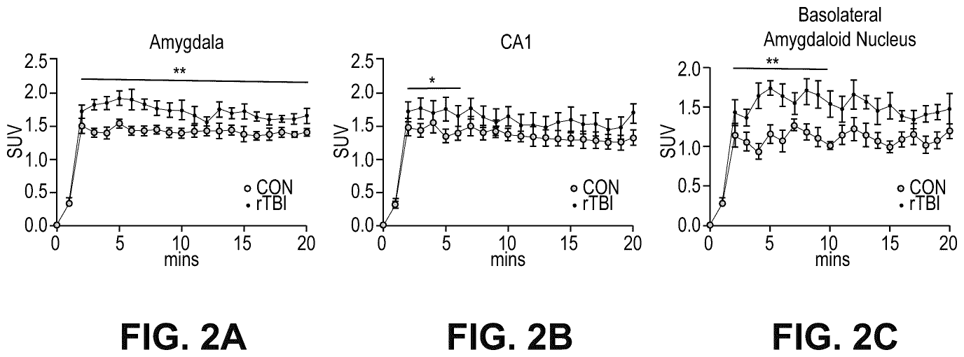

FIG. 2A is a graph of Quantitative Standard uptake values (SUV) for translocator protein (TSPO) radioligand in the amygdala versus time (minutes) comparing control rats (bottom, large circle) with rTBI rats (top, small circle) (n=6 per group) (Time: F(20,200)=110.6, p<0.0001; Treatment: (1, 10)=7.771, p=0.0192; Interaction: (20, 200)=2.321, p=0.0017) two-way-RM ANOVA.

FIG. 2B is a graph of SUV for TSPO radioligand in CA1 versus time (minutes) comparing control rats (bottom, large circle) with rTBI rats (top, small circle) (n=6 per group) (Time: F(5,50)=216.4, p<0.0001; Treatment: F (1,10)=1.711, p=0.2202; Interaction: F(5,50)=2.614, p=0.0355) two-way-RM ANOVA.

FIG. 2C is a graph of SUV for TSPO radioligand in the Basolateral amygdaloid nucleus versus time (minutes) comparing control rats (bottom, large circle) with rTBI rats (top, small circle) (n=6 per group) (Time: F(10,100)=4 9.67, p<0.0001; Treatment: F(1,10)=13.97, p=0.0039; Interaction: F(10,100)=2.605, p=0.0075). In FIGS. 2A-2C, all data are mean±SEM, *p<0.05, **<0.01, ***<0.01, ****<0.001.

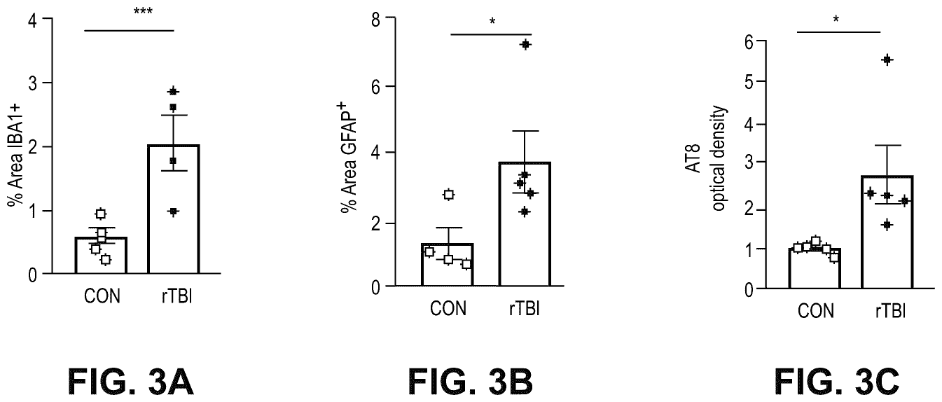

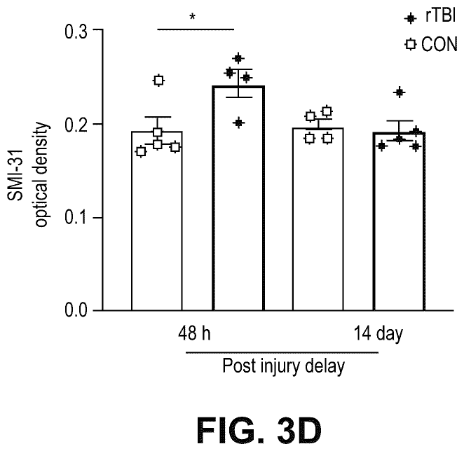

FIG. 3A is a bar graph of Histological quantitation (% Area) for IBA1+ microglia within CA1, comparing control rats (left bar) to rTBI rats (right bar), p=0.0083 Student's t-test. (n=4-5 per group).

FIG. 3B is a bar graph of Histological quantitation (% Area) for GFAP+ astrocytes within CA1 comparing control rats (left bar) to rTBI rats (right bar), p=0.0317 Mann Whitney test (p=0.0300, Shapiro-Wilk) (n=4-5 per group).

FIG. 3C is a bar graph of Histological quantification of AT8 optical density in CA1 at 6 months post injury, comparing control rats (left bar) to rTBI rats (right bar), p=0.0210 Student's t-test (n=5 per group).

FIG. 3D is a bar graph of Histological quantification of SMI-31 optical density at 48 hours and 14 days post injury comparing control rats (left bar) to rTBI rats (right bar). (n=4-5 per group) (48 h Con-TBI, p=0.0409 Student's t-test). In FIGS. 3A-3D, all data are mean±SEM, *p<0.05, **<0.01, ***<0.01, ****<0.001.

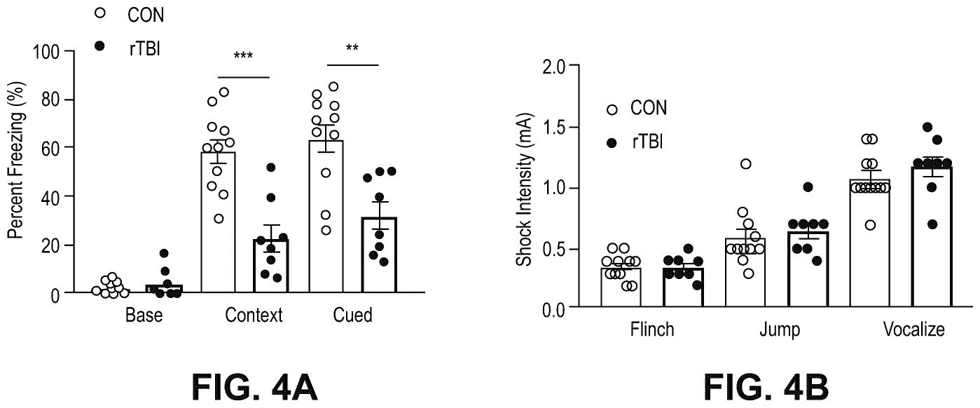

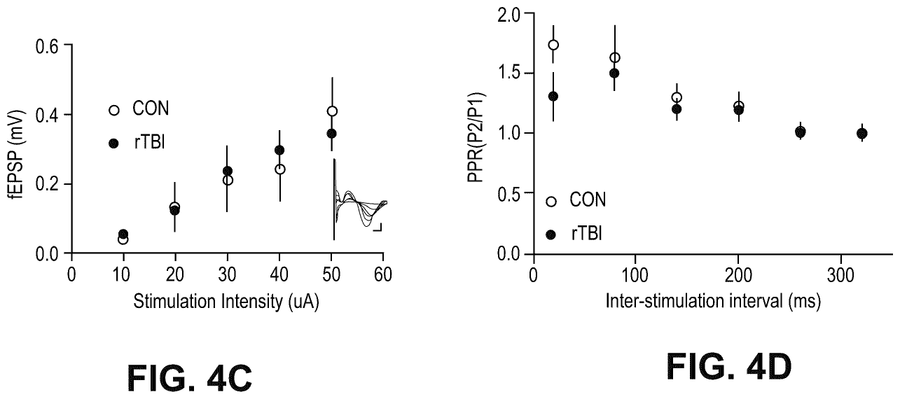

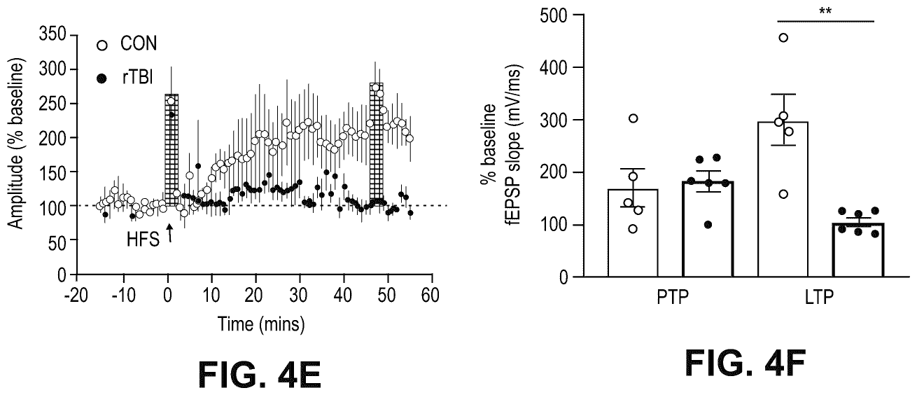

FIGS. 4A-4F are graphs of data relating to behavioral and neurophysiological consequences of rTBI (control rats represented by open circles, rTBI rats represented by closed, smaller circles). All data are means±SEM, *p<0.05, **<0.01, ***<0.001. FIG. 4A is a bar graph of fear conditioning freezing rates (%) for baseline, Contextual, p=0.0001 Student's t-test, and Cued, p=0.0018 Student's t-test fear learning (n=8-11 per group). FIG. 4B is a bar graph of shock sensitivity thresholds (shock intensity, mA) to flinch, jump, or vocalize pain (n=8-11 per group). FIG. 4C is a graph of Input-Output curve of CA3-CA1 fEPSP (mV) recordings for different stimulation intensities (μA) (Inset: individual traces for each of the stimulus intensities) (n=5-6 per group). FIG. 4D is a graph of Paired Pulse Ratio (PPR) across inter-stimulus interval (ms) (n=5-6 per group). FIG. 4E Assessment of the effect of rTBI on hippocampal plasticity after HFS (n=5-6 per group). (PTP outlined grey 0-2 min, LTP outlined grey 45-55 min). FIG. 4F is a bar graph summarizing PTP and LTP fEPSP slopes, (n=5-6 per group) p=0.7662, PTP; p=0.0017, LTP Student's t-test.

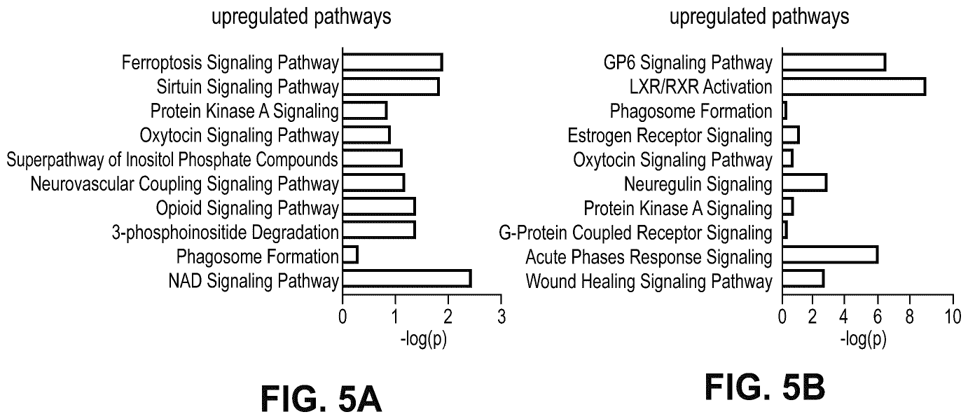

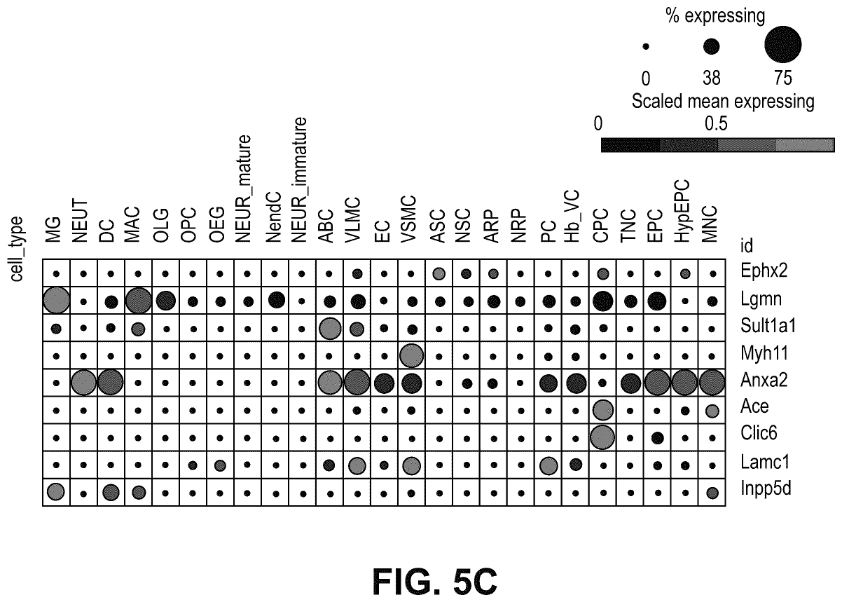

FIG. 5A is a graph of the results from Ingenuity Pathway Analysis of upregulated proteins following rTBI. FIG. 5B is a graph of the results from Ingenuity Pathway analysis of downregulated proteins following rTBI.

FIG. 5C is a table of Cell-type expression profile in brain of differentially expressed proteins assessed from available dataset. Abbreviations: (ABC) Arachnoid barrier cells, (mNEUR) Mature neurons, (ARP) Astrocyte-restricted precursors, (NendC) Neuroendocrine cells, (ASC) Astrocytes, (NEUT) Neutrophils, (CPC) Choroid plexus epithelial cells, (NRP) Neuronal-restricted precursors, (DC) Dendritic cells, (NSC) Neural stem cells, (EC) Endothelial cells, (OEG) Olfactory ensheathing glia (EPC) Ependymocytes, (OLG) Oligodendrocytes, (Hb_VC) Hemoglobin-expressing vascular cells, (OPC) Oligodendrocyte precursor cells, (HypEPC) Hypendymal cells, (PC) Pericytes (ImmN) Immature neurons, (TNC) Tanycytes, (MAC) Macrophages, (VLMC) Vascular and leptomeningeal cells, (MG) Microglia, (VSMC) Vascular smooth muscle cells, (MNC) Monocytes.

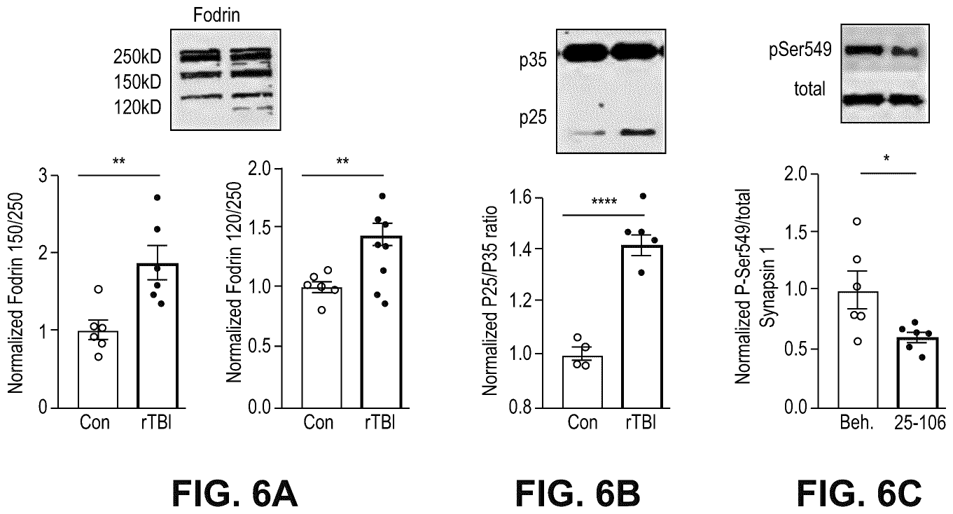

FIG. 6A is a graph of quantitative immunoblot of Fodrin breakdown products via calpain, p=0.0062 (left graph), p=0.0014 (right graph). Student's t-test (n=6 per group).

FIG. 6B is a graph of quantitative immunoblot of p25 generation following rTBI, (n=4 per group) p<0.0001 Student's t-test.

FIG. 6C is a graph of in vivo inhibition of Cdk5 assessed via quantitative immunoblot for P-Ser549/total Synapsin I after treatment with 50 mg/kg 25-106, (n=6 per group) p=0.0305 Student's t-test. In FIGS. 6A-6C, control rats represented by open circles, rTBI rats represented by closed, smaller circles, and all data are means±SEM, *p<0.05, **P<0.01, ***P<0.001, ****P<0.0001.

### DETAILED DESCRIPTION OF THE INVENTION

The testing device disclosed herein can be used to assess the negative consequences of rotational head injury by imparting clinically relevant angular acceleration and deceleration forces to the head of a small mammal, such as a rodent. The testing device induces angular rotational acceleration and deceleration forces that result in injury or insult to the integrity of the intracranial tissues of the brain for the test animal.

As shown in FIG. 1A, an exemplary testing device 100 includes a pneumatic-chain driven pendulum arm 110 attached to a restraint mount, into which a restrained animal can be inserted.

The testing device can include: an animal restraint, a helmet assembly attached to and in pivotal rotation about a central axis of rotation with an animal restraint mount, where a ventral strike plate protrudes from the bottom of the helmet assembly, a pendulum, and an impact fixture in suitable alignment with the ventral strike plate to have a portion thereof, such as an impact bumper contact the strike plate when the pendulum is in motion. The pendulum can be attached to a Pneumatic-chain drive system, which is in fluid communication with a Pneumatic pressure system. One or more sensors are included to measure the effects of the impact on the subject. Optionally one or more sensors are located on the ventral strike plate.

The pendulum can be supported at a desired height to allow for it to swing when a force is applied to the ventral strike plate. For example, the pendulum is pivotally attached at its distal end to a horizontal shaft. The horizontal shaft can be suspended at the desired height by a frame containing one or more frame supports. Thus, the pendulum is able to rotate about the horizontal shaft when a force is applied to the ventral strike plate.

The terms “pendulum” and “pendulum arm” are generally used interchangeably herein.

Exemplary testing devices 100 and parts thereof are shown in FIGS. 1A-1O. The testing device can include an animal restraint 130, a restraint mount 140 configured to receive the animal restraint 130, a helmet assembly 150 which is attached to the restraint mount 140 and in pivotal relationship thereto, a pendulum 110, and an impact fixture 170 (FIGS. 1N and 1O). The pendulum 110 is pivotally attached to and rotates about a horizontal shaft120, both of which are suspended at desired height above a base 102 of the testing device 100. The horizontal shaft 120 is connected and secured at either end, to frame supports 182a, 182b, via securing elements, such as flanged ball bearings 186 (FIG. 1A). For example, a first end of the horizontal shaft 120 is attached to a first frame support 182a and a second end of the horizontal shaft 120 is attached to a second frame support 182b. The first frame support 182a forms the top part of frame 180a and is connected to two frame legs 184a and 184b (FIG. 1A). The second frame support 182b is configured to maintain the horizontal support at the same height as the first frame support. Generally, the first and second frame supports have substantially the same configuration. Optionally, they are mirror images of each other. The second frame support forms the top part of frame 180b with two frame legs 184c and 184d fixedly attached to the second frame support 182b (FIG. 1A). The frames 180a, 180b can be on opposing sides of base 102. The frame legs 184a, 184b, 184c, and 184d are of equal height to permit the pendulum 110 attached to the horizontal shaft 120, to suspend above base 102 and oscillate without touching base 102 (FIG. 1A). The frame legs can be fixedly attached to the base 102 (FIG. 1A).

The animal restraint has a suitable size and shape to receive an animal, such as a rodent, and secure the animal therein. For example, the animal restraint may be in the shape of a hollow tube. However, other geometries are also suitable.

As shown in FIG. 1B, the animal restraint 130 may be in the shape of a hollow tube having a suitable size and shape to fit a rodent therein (also referred to herein as a “rodent restraint”), and optionally contains one or more latches 132, and one or more hinges 134 to allow for opening the rodent restraint to insert the rodent therein, and then closing the rodent restraint 130 to secure the rodent therein. The rodent restraint 130 also includes insertion screws 136a, 136b, 136c or a similar element configured to insert into a corresponding receiving element, such as a slot 144, in the restraint mount 140 (the slot 144 and restraint mount 140 are shown in FIG. 1C). Optionally, one or more insertion screws 136a, 136b protrude from a first side 138b of the rodent restraint 130, and one or more additional insertion screws 136c protrude from second side 138a, optionally where the second side 138a is substantially opposite the first side 138b, of the rodent restraint 130. As shown in FIG. 1C, restraint mount 140 contains one or more slots 144 having a suitable sized opening to receive the insertion screws 136a, 136b, 136c of the rodent restraint 130. Optionally, following insertion into the slot 144 or other receiving element on the restraint mount 140, the insertion screws 136a, 136b can be further secured in place using one or more wing nuts or a similar removable fixation element.

When the rodent is in the animal restraint and the animal restraint is located inside and attached to the restraint mount, the head of the rodent is in contact with the helmet assembly.

As shown in FIG. 1O, the helmet assembly 150 includes a ventral strike plate 152, protruding from the bottom of the helmet assembly. The helmet assembly 150 also includes a head clamp 154, configured to secure the rodent's head therein in a particular orientation. The head clamp 154 can attach to the sides of the rodent's head or the top or bottom of the rodent's head. The head clamp 154 can also include a clamp knob or other device for lowering and raising the head clamp into the desired position to secure (and later to release) the rodent's head in a desired orientation inside the head assembly.

In use, a rodent is placed in an animal restraint 130 inserted into a restraint mount 140 at the end 112 of the pendulum 110 and attached to the freely rotating helmet assembly 150 attached to the restraint mount 140 via a helmet rotation joint 142, permitting pure coronal plane head rotation (FIGS. 1N and 1O). The helmet rotation joint 142 is located at one end of a helmet bracket 146, which is fixedly attached to the restraint mount 140. The helmet rotation joint 142 is attached to the helmet bracket 146 and provides a central axis of rotation for the helmet assembly 150 (FIG. 1O). The helmet rotation joint 142 may be formed of a pin or a bearing and a short round tab or peg that is able to rotate about the central axis of rotation, which in turn allows the rodent's head to rotate about the coronal plane when the rodent is in the device (FIG. 1O). Optionally, restraint mount 140 includes a stop rotation bar 148 to prevent the rodent's head from over-rotating, such as beyond 90° (FIG. 1C).

In use, a rodent, such as a mouse or rat, is placed in animal restraint 130, with the body of the rodent being secured in the animal restraint. The animal restraint 130 is inserted into the fixed restraint mount 140 and secured thereto (FIGS. 1C and 1O). The rodent head is inserted into the helmet assembly 150 by placing the rodent head in an open region within the assembly and securing the rodent head in place by attaching the head clamp 154 to the top or against one or more sides of the rodent head (FIGS. 1C, 1O, and 1G). The helmet assembly 150 can be attached to a helmet bracket 146 via a helmet rotation joint 142. The helmet rotation joint 142 is located on the end of the helmet bracket 146. The helmet bracket is fixedly attached to the restraint mount 140. The helmet rotation joint 142 is capable of rotating about a central axis of rotation, allowing the rodent's head to rotate about the coronal plane when the rodent is in the device (FIG. 1O). In some forms, the restraint mount 140 includes a stop rotation bar 148 to prevent the rodent's head from over-rotating, such as beyond 90° (FIG. 1C).

The testing device also includes an impact fixture for colliding with the ventral strike plate of the helmet assembly during use. The impact fixture includes an impact bumper that is pivotally mounted on a support base and can rotate between two positions using a rotation pin. Additionally, the support base can include an attachment plate that allows for securing the impact fixture to the base of the testing device.

FIG. 1D shows an exemplary impact fixture 170, configured to impact the ventral strike plate of the helmet system or assembly, to which a rodent's head is attached, with the impact bumper 172 when the testing device 100 is in use. The impact bumper 172 is attached to the support base 176 of the impact fixture 170 and is able to rotate from a first position to a second position about a rotation pin 174 or similar connection element, when a force is applied. The impact fixture 170 can be fixedly attached to the base 102 of the testing device 100.

In use, the impact bumper 172 is placed in a first position, which is substantially upright and protruding from the support base 176 of the impact fixture 170. Then, the pendulum 110, swings towards the impact fixture 170, and the impact bumper 172 impacts the ventral strike plate 152 (see FIG. 1G), which transfers impact momentum between the helmet assembly 150 and impact fixture 170. Following impact, the impact bumper 172 is pushed into a second position, which is typically about 10°-90° offset relative to the first position, for example the second position can be offset from the first position by at least about 15°-30°, about 15°-60°, about 15°-70°, about 15°-90°, about 20°-30°, about 20°-60°, about 20°-70°, about 20°-90° or any range or individual measurement within the listed ranges. Upon impact of the ventral strike plate 152, rotational force is imparted on the subject's i.e., the rodent's head.

FIG. 1G (left side) shows the impact bumper 172 in a first position just prior to impact with the ventral strike plate 152. FIG. 1G (right side) shows the impact bumper 172 in a second position, which rotated about the pin 174 by about 90° or more from the first position into the second position.

In use, the restraint is opened and a rodent is placed therein, then the restraint is closed and the latches, clasps, or other suitable locking mechanism is closed, thereby securing the rodent inside the restraint. The restraint is inserted into the mount, such as by sliding the insertion screws in the slots on each of the side walls of the mount. Optionally, thumb screws or another fixation element(s) are inserted into the slots to secure the insertion screws therein. The head of the rodent is then secured in the helmet assembly which is mounted independently on its own bracket, and is able to rotate relative to the mount body. This allows the head to be secured and to rotate independently of the restraint about an axis.

Optionally, as shown in FIGS. 1A and 1E, when used for testing, the pendulum 110 is rapidly propelled forward using a pneumatic chain drive system 160, which includes a compressed gas (e.g. N₂)-driven two-way solenoid valve 162 (FIG. 1E), that is attached to a chain 164 which is connected to the shaft 120, and thereby drives an air motor inducing rapid chain rotation.

The helmet assembly can contain one or more sensors. As depicted in FIG. 1G, an inertial measurement unit (IMU) 114′ can be mounted on the helmet assembly, or a portion thereof, such as the ventral strike plate 152 located below the rat's head. Accelerometry can be derived from the helmet mounted inertial measurement unit (IMU) 114′, such as IMU model 633, a 6-degrees of freedom transducer incorporating accelerometers and gyroscopes comprised of micro-machined silicon sensors.

IMU data can be transmitted to a computer, such as one loaded with software that facilitates visualization of lab tests and measurements. For example, the software may be LabVIEW. The software can convert the IMU data to acceleration measured in gravitational constants (g values), which are converted to angular acceleration (rad/sec²). Optionally, to validate helmet sensor accelerometry, high speed video telemetry can be used to determine angular speed of the helmet. These values can be compared to those derived from the helmet sensor.

In use, one or more computers operable to receive and process the data is provided, where the computer includes and is able to run software suitable for converting and/or evaluating the data. For example, the software can convert the IMU data to acceleration measured in gravitational constants (g values), which are converted to angular acceleration (rad/sec²).

The computer(s) can be a supercomputer, mainframe computer, minicomputer, a microcomputer such as a desktop, or a mobile computer (also referred to as a mobile device) such as a laptop, netbook, tablet, cellphone or smartphone. The computers generally include at least one processing unit and memory. The processing unit executes computer-executable instructions and may be a real or a virtual processor. In a multi-processing system, multiple processing units execute computer-executable instructions to increase processing power. The memory may be volatile memory (e.g., registers, cache, RAM), non-volatile memory (e.g., ROM, EEPROM, flash memory, etc.), or some combination of the two. In some embodiments, the memory stores software that can execute commands to control the testing device and output data such as angular acceleration. A computer may have additional features e.g., storage, one or more input devices, one or more output devices, and one or more communication connections.

The storage may be removable or non-removable, and includes magnetic disks, magnetic tapes or cassettes, CD-ROMs, CD-RWs, DVDs, or any other medium which may be used to store information and which may be accessed via the computer. In some embodiments, the storage stores instructions for the software controlling the testing device.

In the Example, video telemetry and helmet sensor showed highly overlapping determinates for angular speed (see FIG. 1L). Typical machine runs induced average helmet peak rotational acceleration outputs in LabView averaging 482 Krad/s² with limited variability across five sampling runs of 14.15 Krad/s² (see FIG. 1M). These angular accelerations are consistent with human to animal scaling laws for mild brain injuries [30, 31], suggesting the results of rTBI studies using this model in rodents can be used to translate to human conditions.

### Uses

Persistent memory impairment is a common outcome following TBI. Patients report symptoms including both anterograde and retrograde amnesia, as well as cognitive deficits in attention, processing speed, and executive functioning [41]. These deficits often correlate with structural damage to temporal brain areas [35] and damage within the hippocampal formation following TBI can contribute to long-lasting deficits in memory [42].

The deleterious effects of rTBI include shearing and tearing of axonal tracks, compression of brain tissue comprised of various cell types and critical substructures including synapses, dendrites, axons, white matter, grey matter, and neurovasculature. These injuries may lead to sequela including neuronal injury and death, neuroinflammation, compromise in neurovasculature, disruption of the blood brain barrier, and chronic conditions, such as loss of cognitive function, impaired mood, light sensitivity, headaches, balance impairment, edema, and neurodegenerative disorders, including tauopathies and chronic traumatic encephalopathy.

In use, the device induces angular rotational acceleration and deceleration forces to the test animal's brain, which result in injury or insult to the integrity of the intracranial tissues of the brain.

The device and method described herein can be used to investigate one or more functional and/or physiological consequences of rTBI on hippocampal-dependent memory and plasticity.

The device and method described herein can be used to investigate cognitive dysfunction, including the loss of cognitive function, such as manifested by one or more cognitive deficits.

The testing device described herein can be used to in preclinical screening of therapeutics for treatment of and/or prevention of TBI and/or ameliorating one or more symptoms associated with TBI.

The testing device described herein can be used to in preclinical screening of therapeutics for ameliorating one or more the neuropathological consequences of TBI.

The disclosed devices, systems, and methods can be further understood through the following numbered paragraphs:
1. A device for characterizing, testing the effects of, and/or investigating a therapeutic agent for rotational traumatic brain injury comprising: an animal restraint, wherein the animal's head is able to rotate about the coronal plane following impact.
2. A device for characterizing, testing the effects of, and/or investigating a therapeutic agent for rotational traumatic brain injury, the device containing:
an animal restraint,
a restraint mount, configured to receive the animal restraint,
a helmet assembly attached to the restraint mount, such that the helmet assembly is in pivotal rotation about a central axis of rotation,
a pendulum arm, and
an impact fixture.
3. A device for characterizing, testing the effects of, and/or investigating a therapeutic agent for rotational traumatic brain injury including: an animal restraint, wherein in use the device induces angular rotational acceleration and deceleration forces on the animal's head.
4. The device of paragraph 1 or 3, comprising
an animal restraint,
a restraint mount, configured to receive the animal restraint,
a helmet assembly attached to the restraint mount, such that the helmet assembly is in pivotal rotation about a central axis of rotation,
a pendulum arm, and
an impact fixture.
5. The device of paragraph 2 or 4, wherein the helmet assembly contains a ventral strike plate that extends downwardly therefrom.
6. The device of any one of paragraphs 2 or 4 to 5, wherein the impact fixture is aligned with the ventral strike plate such that a portion of the impact fixture contacts the strike plate when the helmet assembly swings toward and past the impact fixture.
7. The device of any one of paragraphs 2 or 4 to 6, wherein the animal restraint has a suitable size and shape to receive a rodent, such as a rat or mouse.
8. The device of any one of paragraphs 2 or 4 to 7, further including a pneumatic-chain drive system.
9. The device of any one of paragraphs 2 or 4 to 8, further including a pneumatic pressure system.
10. The device of any one of paragraphs 2 or 4 to 9, wherein the impact fixture contains an impact bumper, and wherein the impact bumper is aligned with the ventral strike plate such that the impact bumper contacts the strike plate when the helmet assembly swings toward and past the impact fixture.
11. The device of any one of paragraphs 2 or 4 to 10, wherein the helmet assembly further contains one or more sensors, optionally wherein the one or more sensors are located on or adjacent to the ventral strike plate.
12. The device of any one of paragraphs 2 or 4 to 11, wherein the helmet assembly further comprises a head clamp to secure the rodent's head inside the helmet assembly.
13. The device of any one of paragraphs 2 or 4 to 12, wherein the animal restraint is in the shape of a hollow tube, optionally comprising one or more latches, and one or more hinges to allow for opening and closing the hollow tube.
14. The device of any one of paragraphs 2 or 4 to 13, wherein the restraint mount comprises one or more slots, and wherein the animal restraint further comprises one or more insertion screws configured to fit into the slot in the restraint mount,
optionally wherein at least a first of the insertion screws is located on a first side of the animal restraint, and wherein at least a second of the insertion screws is located on a second side of the animal restraint.
15. The device of any one of paragraphs 11 to 14, wherein the one or more sensors is an inertial measurement unit (IMU).
16. A method for characterizing, testing the effects of, and/or investigating a therapeutic agent for rotational traumatic brain injury including
(i) securing a rodent or other small mammal in the device of any one of paragraphs 1 to 16, and
(ii) propelling the pendulum towards the impact fixture.
17. The method of paragraph 16, wherein following step (ii), a portion of the impact fixture contacts ventral strike plate, and the rodent's head is able to rotate in the coronal plane.
18. The method of paragraph 16 or 17, further comprising step (iii) transmitting data from the one or more sensors to a computer, wherein the computer includes software suitable for converting and/or evaluating the data.

### EXAMPLES

### Animals

Male Sprague Dawley rats (9-10 months old, Envigo) were used for all TBI experiments. All were single housed and maintained on a normal 12 hour day/night cycle. For euthanasia in biochemical experiments, brains were collected via rapid live decapitation in the absence of anesthesia. For histological experiments, rats were euthanized via intraperitoneal injections of an anesthetic mixture of (100 mg/kg) ketamine (10 mg/kg) xylazine. For rTBI procedures, rats were maintained under anesthesia (isoflurane) at 1.5% for the duration of the procedure.

### Rotational Traumatic Brain Injury (TBI) Procedure

All rats were habituated to the room in which brain injuries occurred for 1 hour, anesthetized using 1.5% isoflurane vapor, and sedation was maintained throughout the procedure via a nose-cone within the subject's helmet.

Anesthetized rats were placed in a custom designed harness that was inserted into the base of the pendulum and locked tightly into place. The rat head was fitted into a freely rotational helmet with foam padding to secure head placement and rotation in one direction. Flowing N₂ gas was allowed to fill an Arduino controlled closed solenoid value connected to an air motor and the pendulum was adjusted to a 90° angle raised approximately 84 cm from the base of the machine.

Launch was electronically executed using custom software (LabVIEW) to open the solenoid allowing N₂ gas to propel the pendulum forward towards the ventral strike plate. Helmet impact results in a 90° rotation of the helmet and animal's head in both directions. Rotational forces were derived in LabVIEW from helmet velocity and exported into Excel. Each animal received 10 repetitive impact rotations over a 30 minute period. Controls were maintained in the machine, under anesthesia for 30 minutes with no head rotation. Following injury each animal was assessed for normal locomotion and righting reflex abilities.

### PET/CT Imaging

Noninvasive positron emission tomography-computed tomography (PET/CT) imaging was conducted 7 days post TBI with [18F] DPA-714, a translocator protein (TSPO) radioligand produced at the Cyclotron Facility at the University of Alabama at Birmingham. Rats were injected with 300 μCi (11.1 MBq) of [18F] DPA-714 intravenously. Rats were immediately scanned using a GNEXT small animal PET/CT (Sofie Biosciences) for 30 minutes. Imaged regions of interests (ROIs) within the brain were drawn with CT guidance using VivoQuant (Invicro) and a 3D rat brain atlas (Allen brain atlas) was applied to automatically segment each brain region. The mean, maximum, hotspot, and heterogeneity of standard uptake values (SUVs) were determined using the formula: SUV=[(MBq/mL)×(animal wt. (g))/injected dose (MBq)].

### Histology

Histology was performed as described in Crowe AR, Yue W., “Semi-quantitative Determination of Protein Expression using Immunohistochemistry Staining and Analysis: An Integrated Protocol”, Bio Protoc. Dec. 20 2019; 9(24). Briefly, rats were transcardially perfused with 1× PBS/50 mM NaF followed by 10% formalin fixation. Brains were submerged in 10% formalin and fixed overnight, paraffin embedded and serially sectioned at 5 μm at Bregma level −3.3 to −4.5. Sections were deparaffinized in 100% xylene 3×5 minutes. Following deparaffination, slides were rehydrated using ethanol gradients of 5 minute incubations in 100% ethanol, followed by 95% ethanol, and lastly 75% ethanol before being dipped in water for 1 minute. Following rehydration, slides were incubated in 1×pre-warmed citrate antigen retrieval buffer (Thermo Fisher) and heated for 10 minutes in a pressure cooker followed by 20 minutes of cooling in the citrate buffer. Sections were then permeabilized and blocked in 0.03% Trition X-100/PBS containing 3% goat serum. DAB-stained sections underwent a 10 minute incubation in 10% horseradish peroxidase. Immunohistochemistry was performed using glial fibrillary acidic protein (GFAP) (1:1000; Millipore) and ionized Ca²⁺-binding adapter protein (1:1000; Wako) incubated in 0.3% Tween 20/PBS overnight.

Following 1 hour of PBS washes, fluorescent visualization was performed using secondary antibodies Cy3 anti-rat (1:500), Alexa 488 anti-mouse (1:200) (Jackson Immuno) and imaged using an Olympus BX60 microscope mounted with Olympus DP74 digital camera. DAB-stained sections were incubated with anti-mouse biotin-conjugated secondary (1:500) or anti-rabbit biotin-conjugated secondary (1:500) (Jackson Immuno) followed by PBS washing and 30 minutes incubation with ready-to-use streptavidin peroxidase. DAB staining was performed using DAB substrate Kit (abcam) according to manufacturer's instructions and sections were incubated until brown precipitate was visible (5 minutes, AT8; 1 minute SMI-31). Following DAB staining sections were incubated in hematoxylin counterstain and for 1 minute, washed with water, dehydrated in 100% ethanol (3×5 minutes) and cleared in xylene (3×5 minutes).

For Nissl stains, paraffin embedded sections were deparaffinized and rehydrated in serial ethanol washes (as described above). Slides were then incubated in warmed (50° C.) 0.1% cresyl violet solution (Electron Microscopy Sciences) for 10 minutes. Following staining, Nissl sections were differentiated in 95% ethanol, dehydrated in 100% ethanol, and cleared with 100% Xylene.

All stains were performed using a minimum of 3 sections per animal and 4-6 animals per group. All stains within groups were analyzed using the same background thresholds (0-72; GFAP), (4-110; IBA1), and (72-234; AT8). Following thresholding, percent area of immunofluorescent positive staining was determined using ImageJ particle detection software (Fiji), optical density analysis of DAB stained sections was performed as described in Hossain, et al. “Restoration of CTSD (cathepsin D) and lysosomal function in stroke is neuroprotective”, Autophagy. June 2021; 17(6):1330-1348 and OD was determined by log (255/mean intensity) for 8 bit images. Dark Nissl-stained neurons were detected using (Fiji) multipoint counting tool.

### Ex Vivo Acute Brain Slice Pharmacology

Brain slice pharmacology was performed as described in Umfress, et al., “Systemic Administration of a Brain Permeable Cdk5 Inhibitor Alters Neurobehavior” Front Pharmacol. 2022; 13:863762. Briefly, brains were rapidly decapitated and submerged in ice cold Normal Kreb's solution (125 mM NaCl, 2.5 mM KCl, 1.25 mM NaH2PO4, 25 mM NaHCO3, 1.1 mM MgCl2, 2 mM CaCl2 and 25 mM glucose). Brains were coronally sectioned at 350 μm using a vibratome in regions of interest. Slices were recovered in oxygenated Krebs solution at 30° C. Following recovery, slices where incubated in Kreb's containing pharmacological interventions indicated of NMDA/Glycine (100 μM NMDA, 50 μM Gly, 1 h), 25-106 (10 μM, 1 h). Following treatment, slices were snap frozen in dry ice to terminate treatment. Following NMDA treatment, slices were incubated in 0.125% 2,3,5-Triphenyltetrazolium chloride (TTC) viability stain for 20 minutes. Stained slices were fixed in 4% PFA for 10 min and scanned. TTC viability was assessed by mean intensity of staining and expressed as percentage of control viability.

### Immunoblotting

Immunoblotting was performed as previously described in Crowe AR, Yue W, “Semi-quantitative Determination of Protein Expression using Immunohistochemistry Staining and Analysis: An Integrated Protocol” Bio Protoc. Dec. 20 2019; 9 (24). Briefly, rat brains were rapidly dissected and submerged in a 4° C. solution of PBS/50 mM NaF. Subregions were dissected and snap frozen. Brain regions were subsequently homogenized in 1% SDS/50 mM NaF and sonicated at 40 dB pulses until tissue was completely homogenized. Protein concentrations were determined from lysates via BCA protein assay. Samples were diluted in 4×lysis buffer and proteins were separated by molecular weight via SDS-PAGE. Proteins were then transferred to 0.45 nm nitrocellulose, blocked in Licor Blocking Buffer, and incubated with 1° antibody (Ab) overnight. Membranes were washed with 1xTBS-T and incubated with Licor fluorescent 2° Ab for 1 hour at room temperature.

Proteins expression was visualized using Licor Odyssey CLx membrane scanner. Arbitrary units of florescent intensity for each protein band was quantified using ImageStudio. Phospho-band intensity was normalized to total protein bands, total protein bands were normalized to actin loading controls, and cleavage products were normalized to un-cleaved total protein. Antibodies used include p35/p25 (Cell Signaling Technology), phospho-Ser549 and total Synapsin I (PhosphoSolutions), Fodrin (Enzo Life Sciences), phospho-Thr75-and total DARPP32 (Cell Signaling Technology), EPHx2 (Abcam), Legumain (Cell Signaling), AT8 (Invitrogen), Actin (Invitrogen).

### Neurophysiological Recordings

Neurophysiological studies were conducted in rats 7 days post-injury as described in Hernandez, et al. “Exposure to mild blast forces induces neuropathological effects, neurophysiological deficits and biochemical changes”, Mol Brain. Nov. 9 2018; 11(1):64. Briefly, brains were rapidly dissected in ice-cold artificial cerebrospinal fluid (ACSF; 75 mM sucrose, 87 mM NaCl, 2.5 mM KCl, 1.25 mM NaH₂PO4, 25 mM NaHCO₃, 7 mM MgCl₂, 0.5 mM CaCl₂ and 10 mM glucose). Transverse hippocampal slices (350 μ) were sectioned using a vibratome (Leica Microsystems Inc., VT1000S) in NMDG cutting/recovery solution (N-methyl D-glucamine (100 mM), KCl (2.5 mM), NaH₂PO₄ (1.2 mM), NaHCO₃ (30 mM), HEPES (20 mM), MgSO₄ (10 mM), CaCl₂ (0.5 mM), and glucose (25 mM) at 30° C. (pH 7.3-7.4). After 2 minutes, slices were transferred to HEPES holding solution NaCl (92 mM), KCl (2.5 mM), NaH2CO₃ (30 mM), NaH2PO₄ (1 mM), HEPES (20 mM), D-Glucose (25 mM), MgCl₂ (1 mM), CaCl₂ (1 mM) for 1 hour at 30° C. Slices were allowed to incubate for 30 minutes in recording solution of oxygenated Kreb's (125 mM NaCl, 2.5 mM KCl, 1.25 mM NaH2PO₄, 25 mM NaHCO₃, 1.1 mM MgCl₂, 2 mM CaCl₂ and 25 mM glucose) prior to recording.

Recordings were performed using a Multiclamp 700A amplifier with a Digidata 1322 and pClamp 10 software (Axon, Molecular devices, LLC). Field excitatory postsynaptic potentials (fEPSP) from CA1 were evoked by square current pulses (0.1 ms) at 0.033 Hz with a bipolar stimulation electrode (FHC, Bowdoinham, ME). Stimulus intensity was defined using a stimulus intensity required to induce 50% of the maximum EPSP slope using the input-output curves. The sample intensity was used for paired-pulse ratio (PPR) recordings across different intervals. A stable baseline was recorded for at least 10 minutes prior to high frequency stimulation (HFS, 4 trains, 100 Hz, 1 s duration, separated by 20 s). Post-tetanic potentiation (PTP) was analyzed by taking the average of the slopes from the traces recorded during the first 2 minutes after HFS. LTP was assessed for at least 45-50 minutes following HFS. The PPR values were calculated by dividing the second fEPSP slope by the first fEPSP slope (fEPSP2/fEPSP1). All recordings were performed in the absence of any drug treatment and only 1 or 2 slices were recorded from each individual rat. Data were analyzed with Clampfit 10 software (Axon, Molecular devices, LLC).

### Neurobehavior

Fear conditioning studies were performed as described in Plattner, et al. “The role of ventral striatal cAMP signaling in stress-induced behaviors”, Nat Neurosci. August 2015; 18(8):1094-1100. Briefly, rats were habituated to the behavioral room for 1 hour before experimentation. Rats were placed in fear conditioning chambers (Med Associates) to establish baseline freezing rates between cohorts. The following day rats were subjected to fear conditioning training. Each rat was allowed to freely explore the chamber for 2 minutes followed by a 30 seconds tone terminating in a mild foot shock (0.7 mA). Rats remained in the chamber 2 minutes after shocking before returning to their home cage.

Context-dependent fear memory was assessed 24 hours post-shock training. Rats were re-introduced into the conditioning box for 5 minutes. Freezing responses (motionless except respirations) were recorded using VideoFreeze software. Cued fear conditioning memory was assessed 27 hours post-shock training where rats were allowed to explore a novel context with novel odor (vanilla). The rats were left exploring the novel context for 3minutes without tone followed by a 3 minute period with the training tone playing. Freezing responses (motionless except respirations) were recorded.

Shock sensitivity and nociceptive responses were assessed by returning rats to fear conditioning chambers and evaluating minimal thresholds to induce rat flinching, jumping, and vocalization of pain across adverse stimuli (0-1.5 mA) shocks.

### Discovery Proteomics

Freshly dissected and snap frozen rat hippocampi were sonicated, centrifuged, reduced with DTT, and alkylated with iodoacetamide. Total protein for each sample (10 mg) was trypsin digested, purified over C18 columns (Waters), enriched using the PTMScan Phospho CDK+CDK/MAPK Substrate Motif Antibodies (#9477/#2325 Cell Signaling Technology) and purified over C18 tips as described in Stokes, et al. “Complementary PTM Profiling of Drug Response in Human Gastric Carcinoma by Immunoaffinity and IMAC Methods with Total Proteome Analysis”, Proteomes. Aug. 7 2015; 3(3):160-183. For total proteome analysis an additional 100 mg of each sample was digested with LysC and trypsin and digested samples were purified over C18 tips, labeled with TMT 10-plex reagent (Thermo), bRP fractionated (96 fractions concatenated non-sequentially to 12), and C18 purified for LC-MS/MS as previously described in [111]. LC-MS/MS analysis was performed using an Orbitrap-Fusion Lumos Tribrid mass spectrometer as described in [110,111] with replicate injections of each sample run non-sequentially for the phosphopeptide analysis. Briefly, peptides were separated using a 50 cm×100 μM PicoFrit capillary column packed with C18 reversed-phase resin and eluted with a 90 minute (Phospho) or 150 minute (TMT total proteome) linear gradient of acetonitrile in 0.125% formic acid delivered at 280 nl/min. MS spectra were evaluated by Cell Signaling Technology using Comet and the GFY-Core platform (Harvard University) [112, 113, 114]. Searches were performed against the most recent update of the NCBI Rattus norvegicus database with a mass accuracy of ±20 ppm for precursor ions and 0.02 Da product ions.

Results were filtered to a 1% peptide-level FDR with mass accuracy ±5 ppm on precursor ions and presence of a phosphorylated residue for CDK Substrate enriched samples. TMT total proteome results were further filtered to a 1% protein level false discovery rate. Site localization confidence was determined using AScore [115]. All CDK Substrate quantitative results were generated using Skyline [116] to extract the integrated peak area of the corresponding peptide assignments or in GFY-Core using signal: noise values for each peptide and summing individual signal: noise values for all peptides for a given protein for TMT total proteome data. Accuracy of quantitative data was ensured by manual review in Skyline or in the ion chromatogram files. Quantitative data was normalized across samples using median abundance for CDK Substrate data or sum signal: noise for TMT total proteome data.

### Pathway and Ontology Analysis

Differentially modified phosphoproteins across all groups upregulated ≥1.3-fold change or downregulated ≤−1.3-fold change were subjected to Ingenuity Pathway Analysis (IPA), (Qiagen). Log-transformed p-values of significantly enriched canonical pathways and the phosphorylated/dephosphorylated molecules comprising each pathway were used to construct dot plots of canonical pathways significantly Up/downregulated in each condition. IPA Z-scores were used to predict activation or inhibition of each canonical pathway. Phosphoproteins exhibiting fold changes of greater ≥1.3 or ≤−1.3 were subjected to gene ontology enrichment analysis via open source gene enrichment analysis software, Enrichr [117]. Biological processes and molecular functions from each gene list were derived based and the top ten processes/functions were derived based on adjusted p-values.

### Statistical Analysis

Prior to analysis, all data was examined for normalcy using the Shapiro-Wilk test. In cases of parametric data with normal distributions of two group means Student's t-test were used. In cases of non-parametric comparison of means, the Mann-Whitney test was employed. When comparing more than two group means, one or two-way ANOVAs were used with Holm-Sidak post-hoc tests. When comparing two experimental variables within the same animal, Two-way repeated measures ANOVA was used. For all data, *=p<0.05, **=p<0.01, **=p<0.001, ***=p<0.0001 All statistical analysis was performed using Prism 6 (GraphPad Software, Inc.).

### Results

### In Vivo Imaging-PET/CT Neurpathology Studies

To investigate the effects of rTBI, PET/CT neuropathology studies were conducted. For these studies, a diagnostic molecular probe that is in current clinical use for the detection of the neuroinflammatory marker, translocator protein (TSPO) was used. This probe, [18F] DPA-714 detects activated microglia resulting from neuroinflammation in neurogenerative and neuroinflammatory conditions [32-34].

TSPO imaging revealed differential uptake diffusely throughout the brains of both control and rTBI rats 7 days post-injury (data not shown). Standard uptake values (SUV) were significantly increased in several brain regions including amygdala, hippocampus CA1 layer, and the basolateral amygdaloid nucleus of rTBI rats 7 days post-injury in comparison to controls (see FIGS. 2B-D). Effects were also observed diffusely throughout whole cortex with cortical areas including perirhinal and primary somatosensory cortex showing higher labeling following rTBI (data not shown). The differential labeling was region-specific, as no effect was detectible when signal throughout whole brain was quantitated (data not shown). Also, regions such as hippocampus layer CA3 and cerebellum exhibited no change in response to impact (data not shown). Thus, rTBI caused brain region-specific alterations in this biomarker of neuroinflammation, which is consistent with effects observed in humans [32].

### Immunostaining

In addition to in vivo imaging, immunostaining was used to assess neuroinflammatory effects of rTBI. For these experiments, layers of the hippocampal formation were examined, as rTBI effects within this brain region in humans have been linked to consequent memory impairments 35-37. Indeed, broad increases in microgliosis were detected throughout the CA1 subfield of the hippocampus 48 hours post-injury (see FIG. 2E). Furthermore, rats subjected to rTBI showed increased astrogliosis throughout the CA1 (see FIG. 2F). Chronic consequences of brain injury include tauopathy occurring in

chronic traumatic encephalopathy (CTE) [38]. To assess chronic neuropathological changes following rTBI, rats 6 months post-injury were assessed for increased phospho-Tau (AT8) immunoreactivity.

### Results from rTBI

Rotational injury was observed to induce an increase in AT8 immunoreactivity throughout the brain at 6 months post-injury, particularly in areas surrounding vasculature (images not shown, see FIG. 2G). Increased AT8 staining was also observed, with diffuse staining throughout the neuropil in regions of the CA1 in rTBI rats as compared to age-matched controls (images not shown, see FIG. 2G).

Another pathological hallmark of TBI is axonal injury [39]. To assess any structural damage to axons of the hippocampus, the CA1 subregion was stained for phosphorylated neurofilaments (SMI-31), a marker of axonal injury [40]. An increase in SMI-31 was observed 48 hours after injury, but not 14 days after rTBI (see FIG. 2H).

Together, these in vivo imaging and histological studies demonstrate rotational head injury induces neuroinflammation and axonal damage in acute and sub-acute phases of injury.

### Proteomic Alterations Following rTBI

To ascertain the effects of rTBI on protein levels as a net measure of the balance between expression and degradation global proteomics were conducted to identify mechanisms of rTBI associated with the acute inflammatory state of the hippocampus that was observed a 48 hours post injury. 234 proteins demonstrated increased expression (FC≥1.3) and 250 proteins with decreased expression (FC≤−1.3) in rTBI hippocampus relative to controls. The altered expression observed in the proteomic analysis within rTBI lysates was independently validated for two specific proteins.

Proteomic analysis revealed animals subjected to rTBI displayed a 1.6 FC of Legumain (LGMN) protease following injury. Immunoblot analysis of hippocampal lysates confirmed LGMN is significantly upregulated in expression following injury. Additionally, Epoxide hydroxylase 2 (EPHX2) displayed a FC increase of 3.9 in rTBI rats compared to control rats. Similarly, immunoblot analysis displayed significant increases of EPHX2 in hippocampal lysates following injury.

To identify the intracellular pathways altered via rTBI, ingenuity Pathways Analysis was conducted of the top upregulated and downregulated proteins following rTBI (see FIGS. 5A and 5B). rTBI induced upregulation of known cell death dependent pathways linked to brain injury, such as ferroptosis and phagosome formation [43,44]. Bidirectional alterations were observed in homeostatic signaling pathways, such as protein Kinase A (PKA) and oxytocin signaling pathways, both of which have been observed to mediate injury in stroke and TBI [45,46].

Downregulated pathways after injury included proteins associated with inflammatory response elements, such as acute phase response signaling, were downregulated in the hippocampus following rTBI. Notably, neurotropic signaling such as those involving neuregulin and wound healing pathways were also downregulated following rTBI. Utilizing an open-source single-cell gene expression atlas of the mouse brain, the cell-type expression profiles of key upregulated proteins following rTBI47 were determined (FIG. 5). Differentially expressed following rTBI displayed cell-type specific alterations in numerous cell-types within immune cell classes such as microglia (MG), neutrophils (NEUT), dendritic cells (DC), macrophages (MAC), and monocytes (MNC). Proteomic expression was also altered in many vascular cell lineages such as endothelial cells (EC), vascular smooth muscle cells (VSMC), arachnoid barrier cells (ABC), and vascular and leptomeningeal cells (VLMC). The top differentially expressed proteins assessed showed limited or no expression in cell-types of astrocyte lineage such as astrocytes (ASC) and astrocyte restricted precursors (ARP). Likewise, limited proteomic alterations were observed in mature (NEUR_mature), immature (NEUR_immature) and neuroendocrine (NendC) neuronal cell lineages. These data likely indicate global proteomic expression changes are occurring in response to inflammatory immune states and damaged vasculature following rTBI.

Altogether, these analyses reveal protein expression changes that may mediate aspects of rTBI and highlight cell types and pathways linked to inflammatory responses which may ultimately contribute to the consequences of injury.

### rTBI and Excitotoxicity Evoke Aberrant Cdk5 Activity and Cdk5 Inhibition is Neuroprotective

A brain diffusible Cdk5 inhibitor (25-106) with exponential brain distribution kinetics and 30-fold greater specificity for Cdk5 over other cyclin dependent kinase family members was recently discovered [54].

The testing device and model disclosed herein was used to evaluate whether this inhibitor could have rTBI therapeutic potential by blocking aberrant Cdk5/p25 activity. First, it was verified that 25-106 acts as a Cdk5 inhibitor in brain tissue by assessing the effect of slice treatment with 25-106 on phosphorylation of the Cdk5 reporter, Thr75 DARPP32. Indeed, this site was strongly attenuated in brain slices by 25-106 treatment. Next, the neuroprotective capacity of 25-106 under excitotoxic conditions was assessed.

The neuroprotective effect following Cdk5 inhibition was observed in ex vivo brain slices treated with high concentrations of NMDA/Gly as evidenced by TTC viability staining. These data confirm 25-106 as an inhibitor of Cdk5 in brain tissue and suggest it is neuroprotective in an ex vivo model of excitotoxicity.

To investigate the role of Cdk5/p25 signaling in rTBI, the excitotoxic activation of calpain following rTBI was assessed. As one index, the breakdown of the calpain reporter, Fodrin into cleaved 150 and 120 kDa products 48 hours post-injury was examined [55].

Rats subjected to rTBI displayed an increased activation of calpain protease (see FIG. 6A). This effect corresponded to an increase in the aberrant activation of Cdk5 observed through the production of the excitotoxic cofactor p25 (see FIG. 6B). To assess the functionality of 25-106 in vivo, rats were treated with 50 mg/kg I.P. and brain lysates were blotted for Cdk5 dependent phosphorylation states. 25-106 caused a marked reduction in Cdk5 phosphorylation of Synapsin I 6 h after treatment in vivo (see FIG. 6C). Together, these studies demonstrate aberrant activation of Cdk5 in excitotoxcity and rTBI and suggest 25-106 may be used as a therapeutic inhibitor of Cdk5.

### REFERENCES

1. Capizzi A, Woo J, Verduzco-Gutierrez M. Traumatic Brain Injury: An Overview of Epidemiology, Pathophysiology, and Medical Management. Med Clin North Am. March 2020; 104(2):213-238.
2. Guskiewicz KM, Mihalik JP, Shankar V, et al. Measurement of head impacts in collegiate football players: relationship between head impact biomechanics and acute clinical outcome after concussion. Neurosurgery. December 2007; 61(6):1244-1252; discussion 1252-1243.
3. Mihalik JP, Bell DR, Marshall SW, Guskiewicz KM. Measurement of head impacts in collegiate football players: an investigation of positional and event-type differences. Neurosurgery. December 2007; 61(6):1229-1235; discussion 1235.
4. Zhang L, Yang KH, King Al. Biomechanics of neurotrauma. Neurol Res. March-April 2001; 23(2-3):144-156.
5. McKee AC, Daneshvar DH. The neuropathology of traumatic brain injury. Handb Clin Neurol. 2015; 127:45-66.
6. Riggio S, Wong M. Neurobehavioral sequelae of traumatic brain injury. Mt Sinai J Med. April 2009; 76(2):163-172.
7. Pavlovic D, Pekic S, Stojanovic M, Popovic V. Traumatic brain injury: neuropathological, neurocognitive and neurobehavioral sequelae. Pituitary. June 2019; 22(3):270-282.
8. Arciniegas DB, Held K, Wagner P. Cognitive Impairment Following Traumatic Brain Injury. Curr Treat Options Neurol. January 2002; 4(1):43-57.
9. Cuthbert JP, Harrison-Felix C, Corrigan JD, et al. Epidemiology of adults receiving acute inpatient rehabilitation for a primary diagnosis of traumatic brain injury in the United States. J Head Trauma Rehabil. March-April 2015; 30(2):122-135.
10. Adams JW, Alvarez VE, Mez J, et al. Lewy Body Pathology and Chronic Traumatic Encephalopathy Associated With Contact Sports. J Neuropathol Exp Neurol. Sep. 1 2018; 77(9):757-768.
11. Nortje J, Menon DK. Traumatic brain injury: physiology, mechanisms, and outcome. Curr Opin Neurol. December 2004; 17(6):711-718.
12. Masel BE, DeWitt DS. Traumatic brain injury: a disease process, not an event. J Neurotrauma. August 2010; 27(8):1529-1540.
13. Davis AE. Mechanisms of traumatic brain injury: biomechanical, structural and cellular considerations. Crit Care Nurs Q. November 2000; 23(3):1-13.
14. Povlishock JT, Christman CW. The pathobiology of traumatically induced axonal injury in animals and humans: a review of current thoughts. J Neurotrauma. August 1995; 12(4):555-564.
15. Raghupathi R. Cell death mechanisms following traumatic brain injury. Brain Pathol. April 2004; 14(2):215-222.
16. Chen Y, Herrold AA, Gallagher VT, Reilly JL, Parrish T, Breiter HC. Cutting to the Pathophysiology Chase: Translating Cutting-Edge Neuroscience to Rehabilitation Practice in Sports-Related Concussion Management. J Orthop Sports Phys Ther. November 2019; 49(11):811-818.
17. Greve MW, Zink BJ. Pathophysiology of traumatic brain injury. Mt Sinai J Med. April 2009; 76(2):97-104.
18. Shi K, Zhang J, Dong JF, Shi FD. Dissemination of brain inflammation in traumatic brain injury. Cell Mol Immunol. June 2019; 16(6):523-530.
19. Al-Dahhak R, Khoury R, Qazi E, Grossberg GT. Traumatic Brain Injury, Chronic Traumatic Encephalopathy, and Alzheimer Disease. Clin Geriatr Med. November 2018; 34(4):617-635.
20. Dams-O'Connor K, Guetta G, Hahn-Ketter AE, Fedor A. Traumatic brain injury as a risk factor for Alzheimer's disease: current knowledge and future directions. Neurodegener Dis Manag. October 2016; 6(5):417-429.
21. Gardner RC, Byers AL, Barnes DE, Li Y, Boscardin J, Yaffe K. Mild TBI and risk of Parkinson disease: A Chronic Effects of Neurotrauma Consortium Study. Neurology. May 15 2018; 90(20): e1771-e1779.
22. Iverson GL, Gardner AJ, Shultz SR, et al. Chronic traumatic encephalopathy neuropathology might not be inexorably progressive or unique to repetitive neurotrauma. Brain. Dec. 1 2019; 142(12):3672-3693.
23. Yousuf MA, Tan C, Torres-Altoro MI, et al. Involvement of aberrant cyclin-dependent kinase 5/p25 activity in experimental traumatic brain injury. J Neurochem. July 2016; 138(2):317-327.
24. Kusakawa G, Saito T, Onuki R, Ishiguro K, Kishimoto T, Hisanaga S. Calpain-dependent proteolytic cleavage of the p35 cyclin-dependent kinase 5activator to p25. J Biol Chem. Jun. 2 2000; 275(22):17166-17172.
25. Meyer DA, Richer E, Benkovic SA, et al. Striatal dysregulation of Cdk5 alters locomotor responses to cocaine, motor learning, and dendritic morphology. Proc Natl Acad Sci USA. Nov. 25 2008; 105(47):18561-18566.
26. Barnett DG, Bibb JA. The role of Cdk5 in cognition and neuropsychiatric and neurological pathology. Brain Res Bull. Apr. 25 2011; 85(1-2):9-13.
27. Torres-Altoro MI, Mathur BN, Drerup JM, et al. Organophosphates dysregulate dopamine signaling, glutamatergic neurotransmission, and induce neuronal injury markers in striatum. J Neurochem. October 2011; 119(2):303-313.
28. Meyer DA, Torres-Altoro MI, Tan Z, et al. Ischemic stroke injury is mediated by aberrant Cdk5. J Neurosci. Jun. 11 2014; 34(24):8259-8267.
29. Camins A, Verdaguer E, Folch J, Canudas AM, Pallas M. The role of CDK5/P25 formation/inhibition in neurodegeneration. Drug News Perspect. October 2006; 19(8):453-460.
30. Rowson S, Brolinson G, Goforth M, Dietter D, Duma S. Linear and angular head acceleration measurements in collegiate football. J Biomech Eng. June 2009; 131(6):061016.
31. Viano DC, Hamberger A, Bolouri H, Saljo A. Concussion in professional football: animal model of brain injury—part 15. Neurosurgery. June 2009; 64(6):1162-1173; discussion 1173.
32. Coughlin JM, Wang Y, Munro CA, et al. Neuroinflammation and brain atrophy in former NFL players: An in vivo multimodal imaging pilot study. Neurobiol Dis. February 2015; 74:58-65.
33. Israel I, Ohsiek A, Al-Momani E, et al. Combined [(18) F] DPA-714 micro-positron emission tomography and autoradiography imaging of microglia activation after closed head injury in mice. J Neuroinflammation. Jun. 7 2016; 13(1):140.
34. Werry EL, Bright FM, Piguet O, et al. Recent Developments in TSPO PET Imaging as A Biomarker of Neuroinflammation in Neurodegenerative Disorders. Int J Mol Sci. Jun. 28 2019; 20(13).
35. Arulsamy A, Teng J, Colton H, Corrigan F, Collins-Praino L. Evaluation of early chronic functional outcomes and their relationship to pre-frontal cortex and hippocampal pathology following moderate-severe traumatic brain injury. Behav Brain Res. Aug. 1 2018; 348:127-138.
36. Atkins CM. Decoding hippocampal signaling deficits after traumatic brain injury. Transl Stroke Res. December 2011; 2(4):546-555.
37. Bigler ED, Anderson CV, Blatter DD. Temporal lobe morphology in normal aging and traumatic brain injury. AJNR Am J Neuroradiol. February 2002; 23(2):255-266.
38. Lakis N, Corona RJ, Toshkezi G, Chin LS. Chronic traumatic encephalopathy-neuropathology in athletes and war veterans. Neurol Res. April 2013; 35(3):290-299.
39. Johnson VE, Stewart W, Smith DH. Axonal pathology in traumatic brain injury. Exp Neurol. August 2013; 246:35-43.
40. Anderson KJ, Scheff SW, Miller KM, et al. The phosphorylated axonal form of the neurofilament subunit NF-H (pNF-H) as a blood biomarker of traumatic brain injury. J Neurotrauma. September 2008; 25(9):1079-1085.
41. Rabinowitz AR, Levin HS. Cognitive sequelae of traumatic brain injury. Psychiatr Clin North Am. March 2014; 37(1):1-11.
42. Mathias JL, Mansfield KM. Prospective and declarative memory problems following moderate and severe traumatic brain injury. Brain Inj. April 2005; 19(4):271-282.
43. Kenny EM, Fidan E, Yang Q, et al. Ferroptosis Contributes to Neuronal Death and Functional Outcome After Traumatic Brain Injury. Crit Care Med. March 2019; 47(3):410-418.
44. Yu F, Wang Y, Stetler AR, Leak RK, Hu X, Chen J. Phagocytic microglia and macrophages in brain injury and repair. CNS Neurosci Ther. September 2022; 28(9):1279-1293.
45. Mckay EC, Counts SE. Oxytocin Receptor Signaling in Vascular Function and Stroke. Front Neurosci. 2020; 14:574499.
46. Ponsaerts L, Alders L, Schepers M, et al. Neuroinflammation in Ischemic Stroke: Inhibition of cAMP-Specific Phosphodiesterases (PDEs) to the Rescue. Biomedicines. Jun. 22 2021; 9(7).
47. Ximerakis M, Lipnick SL, Innes BT, et al. Single-cell transcriptomic profiling of the aging mouse brain. Nat Neurosci. October 2019; 22(10):1696-1708.
48. Baracaldo-Santamaria D, Ariza-Salamanca DF, Corrales-Hernandez MG, Pachon-Londono MJ, Hernandez-Duarte I, Calderon-Ospina CA. Revisiting Excitotoxicity in Traumatic Brain Injury: From Bench to Bedside. Pharmaceutics. Jan. 8 2022; 14(1).
49. Blennow K, Hardy J, Zetterberg H. The neuropathology and neurobiology of traumatic brain injury. Neuron. Dec. 6 2012; 76(5):886-899.
50. Hernandez A, Tan C, Plattner F, et al. Exposure to mild blast forces induces neuropathological effects, neurophysiological deficits and biochemical changes. Mol Brain. Nov. 9 2018; 11(1):64.
51. Patrick GN, Zukerberg L, Nikolic M, de la Monte S, Dikkes P, Tsai LH. Conversion of p35 to p25 deregulates Cdk5 activity and promotes neurodegeneration. Nature. Dec. 9 1999; 402(6762):615-622.
52. Cruz JC, Tseng HC, Goldman JA, Shih H, Tsai LH. Aberrant Cdk5 activation by p25 triggers pathological events leading to neurodegeneration and neurofibrillary tangles. Neuron. Oct. 30 2003; 40(3):471-483.
53. Pao PC, Tsai LH. Three decades of Cdk5. J Biomed Sci. Nov. 23 2021; 28(1):79.
54. Umfress A, Singh S, Ryan KJ, et al. Systemic Administration of a Brain Permeable Cdk5 Inhibitor Alters Neurobehavior. Front Pharmacol. 2022; 13:863762.
55. Czogalla A, Sikorski AF. Spectrin and calpain: a ‘target’ and a ‘sniper’ in the pathology of neuronal cells. Cell Mol Life Sci. September 2005; 62(17):1913-1924.
56. Iqbal K, Alonso Adel C, Chen S, et al. Tau pathology in Alzheimer disease and other tauopathies. Biochim Biophys Acta. Jan. 3 2005; 1739(2-3):198-210.
57. Carlson KM, Andresen JM, Orr HT. Emerging pathogenic pathways in the spinocerebellar ataxias. Curr Opin Genet Dev. June 2009; 19(3):247-253.
58. Samuels-Lev Y, O'Connor DJ, Bergamaschi D, et al. ASPP proteins specifically stimulate the apoptotic function of p53. Mol Cell. October 2001; 8(4):781-794.
59. Yong XLH, Zhang L, Yang L, et al. Regulation of NMDA receptor trafficking and gating by activity-dependent CaMKIIalpha phosphorylation of the GluN2A subunit. Cell Rep. Jul. 6 2021; 36(1):109338.
60. Nguyen MD, Lariviere RC, Julien JP. Deregulation of Cdk5 in a mouse model of ALS: toxicity alleviated by perikaryal neurofilament inclusions. Neuron. April 2001; 30(1):135-147.
61. Alvarez-Periel E, Puigdellivol M, Brito V, et al. Cdk5 Contributes to Huntington's Disease Learning and Memory Deficits via Modulation of Brain Region-Specific Substrates. Mol Neurobiol. August 2018; 55(8):6250-6268.
62. Kleiven S. Why Most Traumatic Brain Injuries are Not Caused by Linear Acceleration but Skull Fractures are. Front Bioeng Biotechnol. 2013; 1:15.
63. Armstead WM, Vavilala MS. Improving Understanding and Outcomes of Traumatic Brain Injury Using Bidirectional Translational Research. J Neurotrauma. Nov. 15 2020; 37(22):2372-2380.
64. Sorby-Adams AJ, Vink R, Turner RJ. Large animal models of stroke and traumatic brain injury as translational tools. Am J Physiol Regul Integr Comp Physiol. Aug. 1 2018; 315(2):R165-R190.
65. Rowson S, Duma SM, Beckwith JG, et al. Rotational head kinematics in football impacts: an injury risk function for concussion. Ann Biomed Eng. January 2012; 40(1):1-13.
66. Gennarelli TA, Thibault LE, Adams JH, Graham DI, Thompson CJ, Marcincin RP. Diffuse axonal injury and traumatic coma in the primate. Ann Neurol. December 1982; 12(6):564-574.
67. Stemper BD, Shah AS, Budde MD, et al. Behavioral Outcomes Differ between Rotational Acceleration and Blast Mechanisms of Mild Traumatic Brain Injury. Front Neurol. 2016; 7:31.
68. Namjoshi DR, Cheng WH, McInnes KA, et al. Merging pathology with biomechanics using CHIMERA (Closed-Head Impact Model of Engineered Rotational Acceleration): a novel, surgery-free model of traumatic brain injury. Mol Neurodegener. Dec. 1 2014; 9:55.
69. Sauerbeck AD, Fanizzi C, Kim JH, et al. modCHIMERA: a novel murine closed-head model of moderate traumatic brain injury. Sci Rep. May 16 2018; 8(1):7677.
70. Willis EF, MacDonald KPA, Nguyen QH, et al. Repopulating Microglia Promote Brain Repair in an IL-6-Dependent Manner. Cell. Mar. 5 2020; 180(5):833-846 e816.
71. Witcher KG, Bray CE, Chunchai T, et al. Traumatic Brain Injury Causes Chronic Cortical Inflammation and Neuronal Dysfunction Mediated by Microglia. J Neurosci. Feb. 17 2021; 41(7):1597-1616.
72. Breen PW, Krishnan V. Recent Preclinical Insights Into the Treatment of Chronic Traumatic Encephalopathy. Front Neurosci. 2020; 14:616.
73. Tomaiuolo F, Carlesimo GA, Di Paola M, et al. Gross morphology and morphometric sequelae in the hippocampus, fornix, and corpus callosum of patients with severe non-missile traumatic brain injury without macroscopically detectable lesions: a T1 weighted MRI study. J Neurol Neurosurg Psychiatry. September 2004; 75(9):1314-1322.
74. Goldstein LE, Fisher AM, Tagge CA, et al. Chronic traumatic encephalopathy in blast-exposed military veterans and a blast neurotrauma mouse model. Sci Transl Med. May 16 2012; 4(134):134ra160.
75. Perez EJ, Cepero ML, Perez SU, Coyle JT, Sick TJ, Liebl DJ. EphB3 signaling propagates synaptic dysfunction in the traumatic injured brain. Neurobiol Dis. October 2016; 94:73-84.
76. Titus DJ, Wilson NM, Freund JE, et al. Chronic Cognitive Dysfunction after Traumatic Brain Injury Is Improved with a Phosphodiesterase 4B Inhibitor. J Neurosci. Jul. 6 2016; 36(27):7095-7108.
77. Werner C, Engelhard K. Pathophysiology of traumatic brain injury. Br J Anaesth. July 2007; 99(1):4-9.
78. Lee MS, Kwon YT, Li M, Peng J, Friedlander RM, Tsai LH. Neurotoxicity induces cleavage of p35 to p25 by calpain. Nature. May 18 2000; 405(6784):360-364.
79. Saatman KE, Creed J, Raghupathi R. Calpain as a therapeutic target in traumatic brain injury. Neurotherapeutics. January 2010; 7(1):31-42.
80. Buki A, Farkas O, Doczi T, Povlishock JT. Preinjury administration of the calpain inhibitor MDL-28170 attenuates traumatically induced axonal injury. J Neurotrauma. March 2003; 20(3):261-268.
81. Czeiter E, Buki A, Bukovics P, et al. Calpain inhibition reduces axolemmal leakage in traumatic axonal injury. Molecules. Dec. 9 2009; 14(12):5115-5123.
82. Kawamura M, Nakajima W, Ishida A, Ohmura A, Miura S, Takada G. Calpain inhibitor MDL 28170 protects hypoxic-ischemic brain injury in neonatal rats by inhibition of both apoptosis and necrosis. Brain Res. Mar. 10 2005; 1037(1-2):59-69.
83. Meyer D, Altoro MI, Tan Z, et al. Ischemic stroke is mediate by aberrant aberrant Cdk5. J. Neurosci. 2014; 34:8259-8267.
84. Slevin M, Krupinski J. Cyclin-dependent kinase-5 targeting for ischaemic stroke. Curr Opin Pharmacol. November 17 2008.
85. Pozo K, J.A. B. The emerging role of Cdk5 in cancer: A novel cancer therapy target. Trends in Cancer. 2016; 2:606-618.
86. Bk B, Skuntz S, Prochazkova M, et al. Overexpression of the Cdk5 inhibitory peptide in motor neurons rescue of amyotrophic lateral sclerosis phenotype in a mouse model. Hum Mol Genet. Oct. 1 2019; 28(19):3175-3187.
87. Tsai LH, Lee MS, Cruz J. Cdk5, a therapeutic target for Alzheimer's disease? Biochim Biophys Acta. Mar. 11 2004; 1697(1-2):137-142.
88. Wei FY, Tomizawa K. Cyclin-dependent kinase 5 (Cdk5): a potential therapeutic target for the treatment of neurodegenerative diseases and diabetes mellitus. Mini Rev Med Chem. October 2007; 7(10):1070-1074.
89. Binukumar BK, Shukla V, Amin ND, et al. Peptide TFP5/TP5 derived from Cdk5 activator P35 provides neuroprotection in the MPTP model of Parkinson's disease. Mol Biol Cell. Dec. 1 2015; 26(24):4478-4491.
90. Plassman BL, Havlik RJ, Steffens DC, et al. Documented head injury in early adulthood and risk of Alzheimer's disease and other dementias. Neurology. Oct. 24 2000; 55(8):1158-1166.
91. Trabold R, Eros C, Zweckberger K, et al. The role of bradykinin B(1) and B(2) receptors for secondary brain damage after traumatic brain injury in mice. J Cereb Blood Flow Metab. January 2010; 30(1):130-139.
92. Beauchamp K, Mutlak H, Smith WR, Shohami E, Stahel PF. Pharmacology of traumatic brain injury: where is the “golden bullet”? Mol Med. November-December 2008; 14(11-12):731-740.
93. Kirmani BF, Robinson DM, Fonkem E, Graf K, Huang JH. Role of Anticonvulsants in the Management of Posttraumatic Epilepsy. Front Neurol. 2016; 7:32.
94. Wakai A, McCabe A, Roberts I, Schierhout G. Mannitol for acute traumatic brain injury. Cochrane Database Syst Rev. Aug. 5 2013 (8): CD001049.
95. Levin H, Troyanskaya M, Petrie J, et al. Methylphenidate Treatment of Cognitive Dysfunction in Adults After Mild to Moderate Traumatic Brain Injury: Rationale, Efficacy, and Neural Mechanisms. Front Neurol. 2019; 10:925.
96. Silverberg ND, Panenka WJ. Antidepressants for depression after concussion and traumatic brain injury are still best practice. BMC Psychiatry. Mar. 27 2019; 19(1):100.
97. Bergold PJ. Treatment of traumatic brain injury with anti-inflammatory drugs. Exp Neurol. January 2016; 275 Pt 3:367-380.
98. Cap AP. CRASH-3: a win for patients with traumatic brain injury. Lancet. Nov. 9 2019; 394(10210):1687-1688.
99. Bolton AN, Saatman KE. Regional neurodegeneration and gliosis are amplified by mild traumatic brain injury repeated at 24-hour intervals. J Neuropathol Exp Neurol. October 2014; 73(10):933-947.
100. Wang Y, Liu Y, Nham A, et al. Calpain-2 as a therapeutic target in repeated concussion-induced neuropathy and behavioral impairment. Sci Adv. July 2020; 6(27).
101. Zhang BL, Chen X, Tan T, et al. Traumatic brain injury impairs synaptic plasticity in hippocampus in rats. Chin Med J (Engl). March 2011; 124(5):740-745.
102. Luo Y, Zou H, Wu Y, Cai F, Zhang S, Song W. Mild traumatic brain injury induces memory deficits with alteration of gene expression profile. Sci Rep. Sep. 7 2017; 7(1):10846.
103. Mayeux JP, Teng SX, Katz PS, Gilpin NW, Molina PE. Traumatic brain injury induces neuroinflammation and neuronal degeneration that is associated with escalated alcohol self-administration in rats. Behav Brain Res. Feb. 15 2015; 279:22-30.
104. Bolte AC, Dutta AB, Hurt ME, et al. Meningeal lymphatic dysfunction exacerbates traumatic brain injury pathogenesis. Nat Commun. Sep. 10 2020; 11(1):4524.
105. Lakhan SE, Kirchgessner A. Chronic traumatic encephalopathy: the dangers of getting “dinged”. Springerplus. 2012; 1:2.
106. Umfress A, Speed HE, Tan C, et al. Neuropathological Effects of Chemotherapeutic Drugs. ACS Chem Neurosci. Aug. 18 2021; 12(16):3038-3048.
107. Crowe AR, Yue W. Semi-quantitative Determination of Protein Expression using Immunohistochemistry Staining and Analysis: An Integrated Protocol. Bio Protoc. Dec. 20 2019; 9(24).
108. Hossain MI, Marcus JM, Lee JH, et al. Restoration of CTSD (cathepsin D) and lysosomal function in stroke is neuroprotective. Autophagy. June 2021; 17(6):1330-1348.
109. Plattner F, Hayashi K, Hernandez A, et al. The role of ventral striatal CAMP signaling in stress-induced behaviors. Nat Neurosci. August 2015; 18(8):1094-1100.
110. Stokes MP, Farnsworth CL, Gu H, et al. Complementary PTM Profiling of Drug Response in Human Gastric Carcinoma by Immunoaffinity and IMAC Methods with Total Proteome Analysis. Proteomes. Aug. 7 2015; 3(3):160-183.
111. Possemato AP, Paulo JA, Mulhern D, Guo A, Gygi SP, Beausoleil SA. Multiplexed Phosphoproteomic Profiling Using Titanium Dioxide and Immunoaffinity Enrichments Reveals Complementary Phosphorylation Events. J Proteome Res. Apr. 7 2017; 16(4):1506-1514.
112. Eng JK, Jahan TA, Hoopmann MR. Comet: an open-source MS/MS sequence database search tool. Proteomics. January 2013; 13(1):22-24.
113. Huttlin EL, Jedrychowski MP, Elias JE, et al. A tissue-specific atlas of mouse protein phosphorylation and expression. Cell. Dec. 23 2010; 143(7):1174-1189.
114. Villen J, Beausoleil SA, Gerber SA, Gygi SP. Large-scale phosphorylation analysis of mouse liver. Proc Natl Acad Sci USA. Jan. 30 2007; 104(5):1488-1493.
115. Beausoleil SA, Villen J, Gerber SA, Rush J, Gygi SP. A probability-based approach for high-throughput protein phosphorylation analysis and site localization. Nat Biotechnol. October 2006; 24(10):1285-1292.
116. Maclean B, Tomazela DM, Shulman N, et al. Skyline: an open source document editor for creating and analyzing targeted proteomics experiments. Bioinformatics. Apr. 1 2010; 26(7):966-968.
117. Chen EY, Tan CM, Kou Y, et al. Enrichr: interactive and collaborative HTML5 gene list enrichment analysis tool. BMC Bioinformatics. Apr. 15 2013; 14:128.

We claim:

1. A device for characterizing, testing the effects of, and/or investigating a therapeutic agent for rotational traumatic brain injury comprising: an animal restraint, wherein in use the device induces angular rotational acceleration and deceleration forces on the animal's head and wherein the animal's head is able to rotate about the coronal plane following impact.

2. The device of claim 1, wherein the animal restraint has a suitable size and shape to contain a rodent therein.

3. A device for characterizing, testing the effects of, and/or investigating a therapeutic agent for rotational traumatic brain injury comprising
an animal restraint,
a restraint mount, configured to receive the animal restraint,
a helmet assembly attached to the restraint mount, such that the helmet assembly is in pivotal rotation about a central axis of rotation,
a pendulum, and
an impact fixture.

4. The device of claim 3, wherein the helmet assembly comprises a ventral strike plate that extends downwardly therefrom.

5. The device of claim 3, wherein the impact fixture is aligned with the ventral strike plate such that a portion of the impact fixture contacts the ventral strike plate when the helmet assembly swings toward and past the impact fixture.

6. The device of claim 3, wherein the animal restraint has a suitable size and shape to receive a rodent, such as a rat or mouse.

7. The device of claim 3, further comprising a pneumatic-chain drive system.

8. The device of claim 3, further comprising a pneumatic pressure system.

9. The device of claim 3, wherein the impact fixture comprises as an impact bumper, and wherein the impact bumper is aligned with the ventral strike plate such that the impact bumper contacts the strike plate when the helmet assembly swings toward and past the impact fixture.

10. The device of claim 3, wherein the helmet assembly further comprises one or more sensors, optionally wherein the one or more sensors are located on or adjacent to the ventral strike plate.

11. The device of claim 4, wherein the helmet assembly further comprises a head clamp to secure the rodent's head inside the helmet assembly.

12. The device of claim 3, wherein the animal restraint is in the shape of a hollow tube, optionally comprising one or more latches, and one or more hinges to allow for opening and closing the hollow tube.

13. The device of claim 3, wherein the restraint mount comprises one or more slots, and wherein the animal restraint further comprises one or more insertion screws configured to fit into the slot in the restraint mount,
optionally wherein at least a first of the insertion screws is located on a first side of the animal restraint, and wherein at least a second of the insertion screws is located on a second side of the animal restraint.

14. The device of claim 10, wherein the one or more sensors is an inertial measurement unit (IMU).

15. A method for characterizing, testing the effects of, and/or investigating a therapeutic agent for rotational traumatic brain injury comprising
(i) securing a rodent or other small mammal in the device of claim 3, and
(ii) propelling the pendulum towards the impact fixture.

16. The method of claim 15, wherein the helmet assembly comprises the helmet assembly comprises a ventral strike plate, and wherein following step (ii), a portion of the impact fixture contacts the ventral strike plate, and the rodent's head is able to rotate in the coronal plane.

17. The method of claim 16, wherein the helmet assembly further comprises one or more sensors, optionally wherein the one or more sensors are located on or adjacent to the ventral strike plate, and wherein the method further comprises (iii) transmitting data from the one or more sensors to a computer, wherein the computer includes software suitable for converting and/or evaluating the data.
